SEQUENCE LISTING (36 sequences)

SEQ ID NO: 1 - protein, 120 aa, synthetic construct
  REGION 1..120: note = Description of Artificial Sequence: Syntheticpolypeptide
QVQLKQSGPGLVQPSQSLSITCTVSGFSLTNYGVHWVRQSPGKGLEWLGVIWRGGSTDYNAAFMSRLSITKDNSKSQVFFKMNSLQADDTAIYFCALRGNYVHYYLMDYWGQGTSVTVSS

SEQ ID NO: 2 - protein, 107 aa, synthetic construct
  REGION 1..107: note = Description of Artificial Sequence: Syntheticpolypeptide
DIQVTQTPSSLSASLGDRVTISCRASQDISNYLNWYQQKPDGTVKLLIYYTSRLHSGVPSRFSGSGSGTDYSLTISNLDQEDIATYFCQQGNTLPPTFGGGTNLEIK

SEQ ID NO: 3 - protein, 116 aa, synthetic construct
  REGION 1..116: note = Description of Artificial Sequence: Syntheticpolypeptide
EVQLQESGPGLAKPSQTLSLTCSVTGYSITSNYWNWIRKFPGNKLEYMGYISFSGSTYYNPSLKSRISITRDTSKNQYYLQLNSVTTEDTATYYCARWNYYAMDYWGQGTSVTVSS

SEQ ID NO: 4 - protein, 107 aa, synthetic construct
  REGION 1..107: note = Description of Artificial Sequence: Syntheticpolypeptide
DFLLTQSPAILSVSPGERVSFSCRASQSIGTNIHWYQQRTNGSPRLLIKFASESISGIPSRFSGSGSGTDFTLTINSVESEDIADYYCQQSNSWPYTFGGGTKLEIK

SEQ ID NO: 5 - protein, 112 aa, synthetic construct
  REGION 1..112: note = Description of Artificial Sequence: Syntheticpolypeptide
QVQLQQSGAELMKPGASVKLSCKATGYTFTGYWIEWVKQRPGHGLEWIGEILPGNGSTNCNEKFKGKATFTATTSSNTAYMQLSSLTTEDSAIYYCTTVSYWGQGTTLTVSS

SEQ ID NO: 6 - protein, 112 aa, synthetic construct
  REGION 1..112: note = Description of Artificial Sequence: Syntheticpolypeptide
DVVMTQTPLSLPVSLGDQASISCRSSQSLVHSNGNTYLHWYLQKPGQSPNLLIYKVSNRFSGVPDRFTGSGSGTDFTLKISRVEAEDLGVYFCSQSTHVPFTFGSGTKLEIK

SEQ ID NO: 7 - protein, 8 aa, synthetic construct
  REGION 1..8: note = Description of Artificial Sequence: Syntheticpeptide
GFSLTNYG

SEQ ID NO: 8 - protein, 7 aa, synthetic construct
  REGION 1..7: note = Description of Artificial Sequence: Syntheticpeptide
IWRGGST

SEQ ID NO: 9 - protein, 14 aa, synthetic construct
  REGION 1..14: note = Description of Artificial Sequence: Syntheticpeptide
ALRGNYVHYYLMDY

SEQ ID NO: 10 - protein, 6 aa, synthetic construct
  REGION 1..6: note = Description of Artificial Sequence: Syntheticpeptide
QDISNY

SEQ ID NO: 11 - skipped

SEQ ID NO: 12 - protein, 9 aa, synthetic construct
  REGION 1..9: note = Description of Artificial Sequence: Syntheticpeptide
QQGNTLPPT

SEQ ID NO: 13 - protein, 8 aa, synthetic construct
  REGION 1..8: note = Description of Artificial Sequence: Syntheticpeptide
GYSITSNY

SEQ ID NO: 14 - protein, 7 aa, synthetic construct
  REGION 1..7: note = Description of Artificial Sequence: Syntheticpeptide
ISFSGST

SEQ ID NO: 15 - protein, 10 aa, synthetic construct
  REGION 1..10: note = Description of Artificial Sequence: Syntheticpeptide
ARWNYYAMDY

SEQ ID NO: 16 - protein, 6 aa, synthetic construct
  REGION 1..6: note = Description of Artificial Sequence: Syntheticpeptide
QSIGTN

SEQ ID NO: 17 - skipped

SEQ ID NO: 18 - protein, 9 aa, synthetic construct
  REGION 1..9: note = Description of Artificial Sequence: Syntheticpeptide
QQSNSWPYT

SEQ ID NO: 19 - protein, 8 aa, synthetic construct
  REGION 1..8: note = Description of Artificial Sequence: Syntheticpeptide
GYTFTGYW

SEQ ID NO: 20 - protein, 8 aa, synthetic construct
  REGION 1..8: note = Description of Artificial Sequence: Syntheticpeptide
ILPGNGST

SEQ ID NO: 21 - protein, 5 aa, synthetic construct
  REGION 1..5: note = Description of Artificial Sequence: Syntheticpeptide
TTVSY

SEQ ID NO: 22 - protein, 11 aa, synthetic construct
  REGION 1..11: note = Description of Artificial Sequence: Syntheticpeptide
QSLVHSNGNTY

SEQ ID NO: 23 - skipped

SEQ ID NO: 24 - protein, 9 aa, synthetic construct
  REGION 1..9: note = Description of Artificial Sequence: Syntheticpeptide
SQSTHVPFT

SEQ ID NO: 25 - protein, 152 aa, synthetic construct
  REGION 1..152: note = Description of Artificial Sequence: Syntheticpolypeptide
MAVLGLLFCLVTFPSCVLSQVQLKQSGPGLVQPSQSLSITCTVSGFSLTNYGVHWVRQSPGKGLEWLGVIWRGGSTDYNAAFMSRLSITKDNSKSQVFFKMNSLQADDTAIYFCALRGNYVHYYLMDYWGQGTSVTVSSAKTTPPSVYPLAP

SEQ ID NO: 26 - protein, 162 aa, synthetic construct
  REGION 1..162: note = Description of Artificial Sequence: Syntheticpolypeptide
MVSTAQFLGLLLLCFQGTRCDIQVTQTPSSLSASLGDRVTISCRASQDISNYLNWYQQKPDGTVKLLIYYTSRLHSGVPSRFSGSGSGTDYSLTISNLDQEDIATYFCQQGNTLPPTFGGGTNLEIKRADAAPTVSIFPPSSEQLTSGGASVVCFLNNFYPK

SEQ ID NO: 27 - protein, 146 aa, synthetic construct
  REGION 1..146: note = Description of Artificial Sequence: Syntheticpolypeptide
MMVLSLLYLLTALPGILSEVQLQESGPGLAKPSQTLSLTCSVTGYSITSNYWNWIRKFPGNKLEYMGYISFSGSTYYNPSLKSRISITRDTSKNQYYLQLNSVTTEDTATYYCARWNYYAMDYWGQGTSVTVSSAKTTPPSVFPLA

SEQ ID NO: 28 - protein, 162 aa, synthetic construct
  REGION 1..162: note = Description of Artificial Sequence: Syntheticpolypeptide
MVSTAQFLVFLLFWIPASRGDFLLTQSPAILSVSPGERVSFSCRASQSIGTNIHWYQQRTNGSPRLLIKFASESISGIPSRFSGSGSGTDFTLTINSVESEDIADYYCQQSNSWPYTFGGGTKLEIKRADAAPTVSIFPPSSEQLTSGGASVVCFLNNFYPR

SEQ ID NO: 29 - protein, 143 aa, synthetic construct
  REGION 1..143: note = Description of Artificial Sequence: Syntheticpolypeptide
MGWSWVMLFLLSVTAGVHSQVQLQQSGAELMKPGASVKLSCKATGYTFTGYWIEWVKQRPGHGLEWIGEILPGNGSTNCNEKFKGKATFTATTSSNTAYMQLSSLTTEDSAIYYCTTVSYWGQGTTLTVSSAKTTPPSVFPLA

SEQ ID NO: 30 - protein, 166 aa, synthetic construct
  REGION 1..166: note = Description of Artificial Sequence: Syntheticpolypeptide
MKLPVRLLVLMFWIPASTSDVVMTQTPLSLPVSLGDQASISCRSSQSLVHSNGNTYLHWYLQKPGQSPNLLIYKVSNRFSGVPDRFTGSGSGTDFTLKISRVEAEDLGVYFCSQSTHVPFTFGSGTKLEIKRADAAPTVSIFPPSSEQLTSGGASVVCFLNNFYPK

SEQ ID NO: 31 - DNA, 8533 bp, Homo sapiens
  source 1..8533: mol_type = other DNA
attcgcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattaaaaaaaaaaaaaaaaaaaaaaaaaaaaaaaaaaaaaaaaaaaaaaa

SEQ ID NO: 32 - protein, 2639 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 33 - DNA, 8557 bp, Homo sapiens
  source 1..8557: mol_type = other DNA
attcgcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacagtgtcaatcggaggtcacgggctaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattaaaaaaaaaaaaaaaaaaaaaaaaaaaaaaaaaaaaaaaaaaaaaaa

SEQ ID NO: 34 - protein, 2647 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 35 - protein, 9 aa, synthetic construct
  REGION 1..9: note = Description of Artificial Sequence: Syntheticpeptide
AGVAPLQVK

SEQ ID NO: 36 - protein, 14 aa, synthetic construct
  REGION 1..14: note = Description of Artificial Sequence: Syntheticpeptide
YNEQHVPGSPFTAR

(19) United States
(12) Patent Application Publication
(10) Pub. No.: US 2026/0287590 A1
(43) Pub. Date: Sep. 24, 2026

(54) METHODS FOR DIFFERENTIATING BENIGN PROSTATIC HYPERPLASIA FROM PROSTATE CANCER

(71) Applicant: BPGbio, Inc., Framingham, MA (US)

(72) Inventors: Michael Andrew Kiebish, Millis, MA (US); Poornima Tekumalla, Shrewsbury, MA (US); Niven Rajin Narain, Cambridge, MA (US); Rangaprasad Sarangarajan, Boylston, MA (US)

(21) Appl. No.: 19/283,170

(22) Filed: Jul. 28, 2025

Related U.S. Application Data
(63) Continuation of application No. 16/509,808, filed on Jul. 12, 2019, now abandoned.
(60) Provisional application No. 62/697,292, filed on Jul. 12, 2018.

(51) Int. Cl. G01N 33/57555; C12Q 1/6886; G16B 20/00

(52) U.S. Cl. CPC G01N 33/57555; C12Q 1/6886; C12Q 2600/158; G01N 2333/47; G01N 2800/342; G01N 2800/52; G01N 2800/56; G16B 20/00

(57) ABSTRACT

Methods for differentiating between benign prostatic hyperplasia (BPH) and prostate cancer, in a subject, are provided, such methods including the detection of levels of one or more biomarker diagnostic of BPH. The invention also provides a kit for the diagnosis of BPH, without the need for a biopsy.

### REFERENCE TO RELATED APPLICATIONS

This application is continuation of U.S. application Ser. No. 16/509,808, filed on Jul. 1, 2019, which claims the benefit of priority to U.S. Provisional Application No. 62/697,292, filed on Jul. 12, 2018. The entire contents of each of the foregoing applications, including drawings and sequence listings, are incorporated herein by reference.

### SEQUENCE LISTING

The instant application contains a Sequence Listing which has been submitted electronically in XML format and is hereby incorporated by reference in its entirety. Said XML copy, created on May 28, 2026, is named 119992_20103_SL.XML and is 66,006 bytes in size.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND

### A. Field of the Invention

The invention relates generally to use of biomarkers and analytic methods for testing said biomarkers which can be used to diagnose benign prostatic hyperplasia (BPH) without the need for a biopsy. The invention also generally relates to methods for diagnosing, prognosing, monitoring, and treating BPH involving the detection of biomarkers of the invention. The goal of the present invention generally is to distinguish BPH from prostate cancer in a subject, and to avoid unnecessary prostate biopsies in the subject.

### B. Background of the Invention

Prostate cancer is a leading cause of male cancer-related deaths—second only to lung cancer—and afflicts one out of nine men over the age of 65. According to the American Cancer Society, 241,000 new cases of prostate cancer were reported with about 30,000 prostate cancer-related deaths that same year. Although the disease is typically diagnosed in men over the age of 65, its impact is still significant in that the average life span of a man who dies from prostate cancer is reduced by nearly a decade on average. However, if prostate cancer is discovered early, 90% of the cases may be cured with surgery. Once the tumor spreads outside the area of the prostate gland and forms distant metastases, the disease is more difficult to treat. Therefore, early detection is of critical importance to the success of interventional therapies, and for reducing the mortality rate associated with prostate cancer.

Benign prostatic hyperplasia (BPH) is a condition present in many men at risk for prostate cancer, and is known to effect 50% of men aged 51-60, about 70% of men 61-69 and more than 80% of men over age 70. BPH is a condition which is characterized by growth of the prostate gland, which in turn pushes against the urethra and the bladder, causing significant symptoms in the lower urinary tract. These lower urinary tract symptoms (LUTS) often include difficulty urinating, frequent urination, weak force of stream, terminal dribbling, urinary tract infections and erectile dysfunction. In the United States alone, over 2 million men seek medical treatment for BPH each year.

Currently, both BPH and prostate cancer are screened using the same test, including a digital rectal exam (DRE) and/or the measurement of the levels of prostate specific antigen (PSA). However, these approaches have an unacceptably high rate of false-positives for prostate cancer, leading to a prostate biopsy. Indeed, most men (75-80%) with an elevated PSA level turn out to have BPH and do not have prostate cancer as determined by subsequent confirmatory prostate biopsies. There is no known correlation between BPH and prostate cancer, so having BPH does not necessarily indicate a higher likelihood of developing prostate cancer in the future.

Prostate biopsies are very intrusive tests involving obtaining a tissue sample for histopathological analysis from the prostate, which is associated with serious possible complications. Many men are routinely subjected to multiple unnecessary biopsies during their lifetime, thus increasing the risk of serious side effects. Complications-such as infection, internal bleeding, allergic reactions, impotence, and urinary incontinence-induced by needless biopsies and treatments injure many more men than are potentially helped by early detection of cancers.

Currently, 75-80% of men with moderately elevated prostate specific antigen (PSA) levels (between 4-10 ng/mL) and a negative digital rectal exam are subjected to unnecessary biopsies. With about 1.3 million biopsies per year administered in the United States alone, the associated health care costs for the unnecessary biopsies are astronomical. Of the men subjected to biopsies, about one third of them will have moderate or major side effects. In addition to the physical repercussions from undergoing unnecessary biopsies, the procedure has been shown to create significant psychological stress about prostate cancer, even though there is no known correlation between BPH, which is a benign condition, and prostate cancer. For many men with BPH, the cycle of a moderately elevated PSA leading to an unnecessary biopsy occurs multiple times, as do the negative consequences associated with the invasive procedure.

There is a clear and pervasive unmet need for an improved method to distinguish between subjects with BPH and those who have prostate cancer, and thereby avoid unnecessary biopsy to confirm the diagnosis.

### SUMMARY OF THE INVENTION

The present invention based, at least in part, on the discovery that a biomarker panel comprising filamin A (FLNA) level (e.g., serum FLNA concentration), in combination with one or more clinical biomarkers selected from age and prostate volume, is able to differentiate benign prostate hyperplasia (BPH), including lower urinary tract symptoms (LUTS), from prostate cancer in a subject. In one embodiment, a biomarker panel comprising filamin A (FLNA) level (e.g., serum FLNA concentration), with one or more clinical biomarkers selected from age and prostate volume, is able to differentiate BPH, including LUTS, from prostate cancer better than PSA alone. In one embodiment, the subject has had one or more prostate biopsies. The present invention based, also in part, on the discovery that filamin A (FLNA) level (e.g., serum FLNA concentration), in combination with one or more clinical biomarkers selected from age and prostate volume, is able to differentiate BPH, including LUTS, from prostate cancer in a subject, wherein the prostate cancer is characterized as intermediate (e.g., having a Gleason score of between 5 and 7) or aggressive (e.g., having a Gleason score of 8 or above).

The ability to distinguish between BPH, including LUTS, and prostate cancer allows for more accurate diagnosis, for example wherein a subject has had prior screening, e.g., with PSA or digital rectal exam (DRE), and/or is suspected of having an abnormal prostate state such as LUTS, BPH or prostate cancer, and/or has already had one or more biopsy. Screening or monitoring a subject, e.g., a subject who has had prior screening and/or is suspected of having an abnormal prostate state, and/or has already had one or more biopsy, using the biomarker panel described herein, provides for the differentiation between BPH, including LUTS, and prostate cancer and therefore avoids costly, invasive, and potentially harmful unnecessary procedures such as prostate biopsy.

In one aspect, the present invention provides a method for differentiating benign prostatic hyperplasia (BPH) from prostate cancer, comprising: detecting the protein level of Filamin A (FLNA) in a biological sample from the subject; measuring the prostate volume of the subject; analyzing the age of the subject, the prostate volume of the subject, and the protein level of FLNA in the biological sample with a corresponding predetermined threshold value for FLNA level, age and prostate volume; and determining whether the subject has BPH or prostate cancer by comparing the age of the subject, the prostate volume of the subject, and the protein level of FLNA in the biological sample to the corresponding threshold value.

In one embodiment, if the protein level of FLNA, prostate volume, and age of the subject is below the corresponding predetermined threshold value, the subject is diagnosed with BPH, and if the protein level of FLNA, prostate volume, and age of the subject is above the corresponding predetermined threshold value, the subject is diagnosed with prostate cancer.

In one embodiment, the protein level of FLNA is detected using an assay selected from an immunoassay, an enzyme-linked immunosorbent assay (ELISA), or a mass spectrometry assay.

In another embodiment, the protein level of FLNA is detected using immunoprecipitation multiple reaction monitoring (IP-MRM).

In another embodiment, the protein level of FLNA is detected using a binding protein.

In another embodiment, the binding protein is an anti-FLNA antibody.

In another embodiment, the biological sample is selected from the group consisting of blood, serum, plasma, and urine.

In another embodiment, the biological sample is a serum sample.

In another embodiment, the age of the subject is 50 years or older.

In another embodiment, the subject is experiencing lower urinary tract symptoms (LUTS).

In another embodiment, the subject has an enlarged prostate gland as determined by digital rectal examination (DRE).

In another embodiment, the subject does not have an enlarged prostate gland as determined by digital rectal examination (DRE).

In another embodiment, the subject has an elevated prostate specific antigen (PSA) level.

In another embodiment, the subject has a prostate specific antigen level (PSA) of between 4-10 ng/ml.

In another embodiment, the subject has had one or more prostate biopsies.

In another embodiment, the BPH is differentiated from prostate cancer in a subject having an intermediate Gleason score of from 5 to 7.

In another embodiment, BPH is differentiated from prostate cancer in a subject having a high Gleason score of greater than 8.

In another embodiment, the method further comprises administering to the subject diagnosed with BPH a therapeutic treatment for BPH.

In one embodiment, the therapeutic treatment comprises a selective α₁-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

In another aspect, the present invention provides a method for diagnosing benign prostatic hyperplasia (BPH) in a subject, comprising: detecting the protein level of Filamin A (FLNA) in a biological sample from the subject; measuring the prostate volume of the subject; analyzing the age of the subject, the prostate volume of the subject, and the protein level of FLNA in the biological sample with a corresponding predetermined threshold value for FLNA level, age and prostate volume; and determining whether the subject has BPH by comparing the age of the subject, the prostate volume of the subject, and the protein level of FLNA in the biological sample to the corresponding threshold value.

In one embodiment, if the protein level of FLNA, prostate volume, and age of the subject are below the corresponding predetermined threshold value, the subject is diagnosed with BPH.

In one embodiment, the protein level of FLNA is detected using an assay selected from an immunoassay, an enzyme-linked immunosorbent assay (ELISA), or a mass spectrometry assay.

In another embodiment, the immunoassay is immunoprecipitation multiple reaction monitoring (IP-MRM).

In another embodiment, the protein level of FLNA is detected using a binding protein.

In another embodiment, the binding protein is an anti-FLNA antibody.

In another embodiment, the biological sample is selected from the group consisting of blood, serum, plasma, and urine.

In another embodiment, the biological sample is a serum sample.

In another embodiment, the age of the subject is 50 years or older.

In another embodiment, the subject is experiencing lower urinary tract symptoms (LUTS).

In another embodiment, the subject has an enlarged prostate gland as determined by digital rectal examination (DRE).

In another embodiment, the subject does not have an enlarged prostate gland as determined by digital rectal examination (DRE).

In another embodiment, the subject has an elevated prostate specific antigen (PSA) level.

In another embodiment, the subject has a prostate specific antigen level (PSA) of between 4-10 ng/mL.

In another embodiment, the subject has had one or more prostate biopsies.

In another embodiment, the method comprises administering to the subject a therapeutic treatment for BPH.

In another embodiment, the therapeutic treatment comprises a selective α₁-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

In another aspect, the present invention provides a method for treating BPH comprising detecting the protein level of Filamin A (FLNA) in a biological sample from the subject; measuring the prostate volume of the subject; analyzing the age of the subject, the prostate volume of the subject, and the protein level of FLNA in the biological sample with a corresponding predetermined threshold value for FLNA level, age and prostate volume; and determining whether the subject has BPH or prostate cancer by comparing the age of the subject, the prostate volume of the subject, and the protein level of FLNA in the biological sample to the corresponding threshold value, wherein if the subject has BPH, the subject is administered a treatment comprising a selective α₁-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

In some embodiments, the subject is not subjected to a prostate biopsy.

In some embodiments, the protein level of FLNA is detected using an assay selected from an immunoassay, an enzyme-linked immunosorbent assay (ELISA), or a mass spectrometry assay.

In some embodiments, the protein level of FLNA is detected using immunoprecipitation multiple reaction monitoring (IP-MRM).

In some embodiments, the protein level of FLNA is detected using a binding protein.

In some embodiments, the binding protein is an anti-FLNA antibody.

In some embodiments, the biological sample is selected from the group consisting of blood, serum, plasma, and urine.

In some embodiments, the biological sample is a serum sample.

In some embodiments, the age of the subject is 50 years or older.

In some embodiments, the subject is experiencing lower urinary tract symptoms (LUTS).

In some embodiments, the subject has an enlarged prostate gland as determined by digital rectal examination (DRE).

In some embodiments, the subject does not have an enlarged prostate gland as determined by digital rectal examination (DRE).

In some embodiments, the subject has an elevated prostate specific antigen (PSA) level.

In some embodiments, the subject has a prostate specific antigen level (PSA) of between 4-10 ng/mL.

In one aspect, the present invention provides a method for avoiding an unnecessary prostate biopsy in a subject, comprising detecting the protein level of Filamin A (FLNA) in a biological sample from the subject; measuring the prostate volume of the subject; analyzing the age of the subject, the prostate volume of the subject, and the protein level of FLNA in the biological sample with a corresponding predetermined threshold value for FLNA level, age and prostate volume; and determining whether the subject has BPH or prostate cancer by comparing the age of the subject, the prostate volume of the subject, and the protein level of FLNA in the biological sample to the corresponding threshold value, wherein a biopsy is not required if the subject has BPH.

In one embodiment, if the protein level of FLNA, prostate volume, and age of the subject is below the corresponding predetermined threshold value, the subject does not require a prostate biopsy.

In some embodiments, protein level of FLNA is detected using an assay selected from an immunoassay, an enzyme-linked immunosorbent assay (ELISA), or a mass spectrometry assay.

In some embodiments, the protein level of FLNA is detected using immunoprecipitation multiple reaction monitoring (IP-MRM).

In some embodiments, the protein level of FLNA is detected using a binding protein.

In some embodiments, the binding protein is an anti-FLNA antibody.

In some embodiments, the biological sample is selected from the group consisting of blood, serum, plasma, and urine.

In some embodiments, the biological sample is serum.

In some embodiments, the age of the subject is 50 years or older.

In some embodiments, the subject is experiencing lower urinary tract symptoms (LUTS).

In some embodiments, the subject has an enlarged prostate gland as determined by digital rectal examination (DRE).

In some embodiments, the subject does not have an enlarged prostate gland as determined by digital rectal examination (DRE).

In some embodiments, the subject has an elevated prostate specific antigen (PSA) level.

In some embodiments, the subject has a prostate specific antigen level (PSA) of between 4-10 ng/mL.

In some embodiments, the subject has had one or more prostate biopsies.

In some aspects, the present invention provides a method for monitoring a subject suspected of having benign prostate hyperplasia (BPH) or prostate cancer, comprising detecting the protein level of Filamin A (FLNA) in a biological sample from the subject; measuring the prostate volume of the subject; analyzing the age of the subject, the prostate volume of the subject, and the protein level of FLNA in the biological sample with a corresponding predetermined threshold value for FLNA level, age and prostate volume; and monitoring whether the subject has BPH or prostate cancer by comparing the age of the subject, the prostate volume of the subject, and the protein level of FLNA in the biological sample to the corresponding threshold value.

In one embodiment, if the protein level of FLNA, prostate volume, and age of the subject is below the corresponding predetermined threshold value, the subject is diagnosed with BPH, and if the protein level of FLNA, prostate volume, and age of the subject is above the corresponding predetermined threshold value, the subject is diagnosed with prostate cancer.

In some embodiments, the monitoring is performed more than once.

In some embodiments, the monitoring is performed one every month, once every six months, or once every year.

In some embodiments, the protein level of FLNA is detected using an assay selected from an immunoassay, an enzyme-linked immunosorbent assay (ELISA), or a mass spectrometry assay.

In some embodiments, the protein level of FLNA is detected using immunoprecipitation multiple reaction monitoring (IP-MRM).

In some embodiments, the protein level of FLNA is detected using a binding protein.

In some embodiments, the binding protein is an anti-FLNA antibody.

In some embodiments, the biological sample is selected from the group consisting of blood, serum, plasma, and urine.

In some embodiments, the biological sample is a serum sample.

In some embodiments, the age of the subject is 50 years or older.

In some embodiments, the subject is experiencing lower urinary tract symptoms (LUTS).

In some embodiments, the subject has an enlarged prostate gland as determined by digital rectal examination (DRE).

In some embodiments, the subject does not have an enlarged prostate gland as determined by digital rectal examination (DRE).

In some embodiments, the subject has an elevated prostate specific antigen (PSA) level.

In some embodiments, the subject has a prostate specific antigen level (PSA) of between 4-10 ng/mL.

In some embodiments, the subject has had one or more prostate biopsies.

In some aspects, the present invention provides a kit for differentiating benign prostatic hyperplasia (BPH) from prostate cancer in a subject comprising one or more reagents for detecting a level of FLNA in a biological sample and a set of instructions for detecting the level of FLNA in the biological sample, and for differentiating BPH from prostate cancer by analyzing the level of FLNA in the biological sample, the prostate volume of the subject, and the age of the subject.

In some embodiments, the biological sample is obtained from a subject having, suspected of having, or at risk for having a prostate condition.

In some embodiments, the subject has a prostate specific antigen level (PSA) level between 4-10 ng/mL.

In some embodiments, the age of the subject is 50 years or older.

In some embodiments, the subject is experiencing lower urinary tract symptoms (LUTS).

In some embodiments, the subject has an enlarged prostate gland as determined by digital rectal examination (DRE).

In some embodiments, the biological sample is selected from the group consisting of blood, serum, plasma, and urine.

In some embodiments, the biological sample is a serum sample.

In some embodiments, the level of FLNA in the sample is a protein level.

In some embodiments, the one or more reagents comprise an antibody.

In some embodiments, the methods of the invention further comprise a means to detect the antibody.

In some embodiments, the instructions comprise a predetermined threshold value of FLNA level for comparing to the FLNA level in the biological sample from the subject.

In some embodiments, when the FLNA level in the biological sample is determined to be higher than the predetermined threshold, the subject requires a biopsy, and wherein when the FLNA level in the biological is determined to be lower than the predetermined threshold, the subject does not require a biopsy.

In some embodiments, the instructions comprise directions for performing an immunoassay, ELISA, or mass spectrometry assay for detecting the level of FLNA in the biological sample.

In some embodiments, the instructions comprise directions for performing immunoprecipitation multiple reaction monitoring (IP-MRM).

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A-FIG. 1B depict that a biomarker panel comprising FLNA, age, and prostate volume performs better than PSA alone in differentiating patients with benign prostate hyperplasia (BPH) from prostate cancer (PCa). FIG. 1A shows beeswarm and boxplot graphs showing median, interquartile range and distribution of age (years), prostate volume (mL), serum FLNA concentrations (pg/mL, as determined by IPMRM), and serum PSA concentrations (ng/ml) from patients with BPH compared to those with PCa. Data represents n=300 BPH and n=477 PCa. FIG. 1B is a receiver operator characteristics (ROC) curve for the biomarker panel (FLNA, age and prostate volume) which demonstrates better performance in differentiating patients with BPH from PCa compared to PSA alone. Area under the curve (AUC) for the panel is 0.75 vs. 0.52 for PSA. Shaded grey regions indicate standard error.

FIG. 2A-FIG. 2B depict that the biomarker panel comprising FLNA, age, and prostate volume differentiates benign prostate hyperplasia (BPH) from prostate cancer (PCa) in a subset of patients who have had multiple biopsies. FIG. 2A shows beeswarm and boxplot graphs showing median, interquartile range and distribution of age (years), prostate volume (mL), serum FLNA concentrations (pg/mL, as determined by IPMRM), and serum PSA concentrations (ng/ml) from patients who have had multiple biopsies. Data represents n=300 BPH and n=477 PCa. FIG. 2B is a receiver operator characteristics (ROC) curve for the biomarker panel which demonstrates better performance in differentiating patients with BPH from PCa compared to PSA alone. Area under the curve (AUC) for the panel is 0.87 vs. 0.52 for PSA. Shaded grey regions indicate standard error.

FIG. 3A-FIG. 3B depict that the biomarker panel comprising FLNA, age, and prostate volume differentiates benign prostate hyperplasia (BPH) from prostate cancer (PCa) in patients with intermediate and high Gleason Scores. FIG. 3A is a receiver operator characteristics (ROC) curve for the biomarker panel which demonstrates better performance in differentiating patients with BPH from PCa compared to PSA alone in patients with an intermediate Gleason score (5-7). Area under the curve (AUC) for the panel is 0.76 vs. 0.52 for PSA. Shaded grey regions indicate standard error. FIG. 3B is a receiver operator characteristics (ROC) curve for the biomarker panel which demonstrates better performance in differentiating patients with BPH from PCa compared to PSA alone in patients with a high Gleason score (8-10). Area under the curve (AUC) for the panel is 0.74 vs. 0.47 for PSA. Shaded grey regions indicate standard error.

FIG. 4A-FIG. 4B depict that the biomarker panel comprising FLNA, age, and prostate volume differentiates benign prostate hyperplasia (BPH) from prostate cancer (PCa) in patients with a Gleason score of 5-6 or a Gleason score of 7-10. FIG. 4A is a receiver operator characteristics (ROC) curve for the biomarker panel which demonstrates better performance in differentiating patients with BPH from PCa compared to PSA alone in patients with a Gleason score of 5-6. Area under the curve (AUC) for the panel is 0.77 vs. 0.61 for PSA. Shaded grey regions indicate standard error. FIG. 4B is a receiver operator characteristics (ROC) curve for the biomarker panel which demonstrates better performance in differentiating patients with BPH from PCa compared to PSA alone in patients with Gleason score of 7-10. Area under the curve (AUC) for the panel is 0.8 vs. 0.52 for PSA. Shaded grey regions indicate standard error.

FIG. 5A-FIG. 5B are flow charts depicting the current prostate health screening paradigm utilized by both primary care physicians and urologists (FIG. 5A) and exemplary prostate health screening paradigms carried out using the biomarker panel comprising FLNA level in combination with one or more of age and prostate volume (FIG. 5B). As shown in FIG. 5B, the exemplary screening test utilizing the biomarker panel of the invention can differentiate between BPH and prostate cancer, thus avoiding unnecessary biopsies. Where prostate cancer is suspected, a biopsy can be performed, followed by active monitoring with the biomarker panel of the invention when the biopsy results are negative.

### DETAILED DESCRIPTION

### A. Overview

As described herein, the present invention based, at least in part, on the discovery that a biomarker panel comprising filamin A (FLNA) level (e.g., serum FLNA concentration), in combination with one or more clinical biomarkers selected from age and prostate volume, is able to differentiate benign prostate hyperplasia (BPH), including lower urinary tract symptoms (LUTS), from prostate cancer in a subject. In one embodiment, a biomarker panel comprising filamin A (FLNA) level (e.g., serum FLNA concentration), with one or more clinical biomarkers selected from age and prostate volume, is able to differentiate BPH, including LUTS, from prostate cancer better than PSA alone. In one embodiment, the subject has had one or more prostate biopsies. In one embodiment, the subject has had multiple prostate biopsies.

As described herein, the present invention based, also in part, on the discovery that filamin A (FLNA) level (e.g., serum FLNA concentration), in combination with one or more clinical biomarkers selected from age and prostate volume, is able to differentiate BPH, including LUTS, from prostate cancer in a subject, wherein the prostate cancer is characterized as intermediate (e.g., having a Gleason score of between 5 and 7) or aggressive (e.g., having a Gleason score of 8 or above).

The ability to distinguish between BPH and prostate cancer allows for more accurate diagnosis, for example wherein a subject has had prior screening, e.g., with PSA or digital rectal exam (DRE), and/or is suspected of having an abnormal prostate state such as LUTS, BPH or prostate cancer, and/or has already had one or more biopsy. Screening or monitoring a subject, e.g., a subject who has had prior screening and/or is suspected of having an abnormal prostate state, and/or has already had one or more biopsy, using the biomarker panel described herein, provides for the differentiation between BPH and prostate cancer and therefore avoids costly, invasive, and potentially harmful unnecessary procedures such as prostate biopsy.

Accordingly, the invention provides methods for differentiating BPH from prostate cancer in a subject. The invention also provides methods for diagnosing and/or monitoring BPH, including LUTS, in a subject, e.g., a subject suspected of having an abnormal prostate state such as LUTS, BPH, or prostate cancer, and/or an elevated PSA level or an enlarged prostate, or where a subject has already had one or more biopsy. Based on the ability to differentiate BPH from prostate cancer in a subject, the present invention also provides methods for avoiding unnecessary prostate biopsy in a subject (either an initial biopsy or subsequent biopsies). In some embodiments, the present invention provides methods for screening or monitoring a subject who has previously had one or more negative prostate biopsy. The invention also provides methods for treating BPH, including LUTS, in a subject.

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

### B. Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. The term “such as” is used herein to mean, and is used interchangeably, with the phrase “such as but not limited to.”

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. As used herein, “one or more” is understood as each value 1, 2, 3, 4, 5, 6, 7, 8, 9, 10, and any value greater than 10.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, select terms are defined below.

As used herein, the term “sample” refers generally to a limited quantity of something which is intended to be similar to and represent a larger amount of that thing. In the present disclosure, a sample is a collection, fluid, blood, swab, brushing, scraping, biopsy, removed tissue, or surgical resection that is to be tested. In some embodiments, the sample is a bodily fluid such as blood, serum, or plasma. In some embodiments, samples are taken from a subject having or that is believed or suspected of benign prostatic hyperplasia (BPH), LUTS, or prostate cancer (PCa). In some embodiments, a sample is taken from a subject from a subject that has had one or more PSA test, digital rectal examination (DRE) and/or prostate biopsy. In some embodiments, the PSA test results indicate an elevated level of PSA in the subject. In other embodiments, the DRE indicates an enlarged prostate. In other embodiments, the DRE does not indicate an enlarged prostate. In other embodiments, the biopsy does not indicate prostate cancer.

The term “control sample,” “control,” or “normal,” or “reference sample” as used herein, refer to samples with a known presence, absence, or quantity of substance being measured, that is used for comparison against an experimental sample. As used herein, the term refers to any clinically relevant comparative sample, including, for example, a sample from a healthy subject not afflicted with an abnormal prostate state, e.g., LUTS, BPH, or prostate cancer, a sample from a subject afflicted with BPH, a sample from a subject afflicted with prostate cancer, or a sample from a subject from an earlier time point, e.g., prior to treatment, an earlier assessment time point, or at an earlier stage of treatment. In some embodiments, a control sample can be from a subject having an enlarged prostate. In other embodiments, a control sample can be from a subject without an enlarged prostate. In some embodiments, a control sample can be from a subject with an elevated PSA level. In some embodiments, a control sample can be a purified sample, protein, and/or nucleic acid provided with a kit. In some embodiments, such control samples can be diluted, for example, in a dilution series to allow for quantitative measurement of levels of analytes, e.g., markers, in test samples. A control sample may include a sample derived from one or more subjects. A control sample may also be a sample made at an earlier time point from the subject to be assessed. For example, the control sample could be a sample taken from the subject to be assessed before the onset of an abnormal prostate state, e.g., LUTS, BPH, or prostate cancer, at an earlier stage of disease, or before the administration of treatment or of a portion of treatment. In some embodiments, the control sample may also be a sample from an animal model, or from a tissue or cell line derived from the animal model of an abnormal prostate state, e.g., LUTS, BPH, or prostate cancer. In some embodiments, the level of activity or expression of one or more markers (e.g., 1, 2, 3, 4, 5, 6, 7, 8, or 9 or more markers) in a control sample consists of a group of measurements that may be determined, e.g., based on any appropriate statistical measurement, such as, for example, measures of central tendency including average, median, or modal values. In some embodiments, different from a control is statistically significantly different from a control. In some embodiments, any tissue or body fluid sample may be used to detect the absence or presence of an abnormal prostate state, e.g., LUTS, BPH, or prostate cancer. Cystic fluid, saliva, cheek swabs (buccal swabs), hair bulb, blood serum, plasma, and whole blood samples are among the common forms of samples used to obtain such samples. Examples of other samples can include semen, urine, lymph fluid, cerebral spinal fluid, amniotic fluid, skin and surgically excised tissue. One skilled in the art would readily recognize other types of samples and methods of obtaining them. In some embodiments of the methods disclosed herein, any of the methods disclosed herein comprise a step of obtaining a sample from a subject such as a human patient.

As used herein, “changed as compared to a control” sample or subject is understood as having a level of the analyte or diagnostic or therapeutic indicator (e.g., FLNA) to be detected at a level that is statistically different than a sample from a normal, untreated, or abnormal state control sample. Changed as compared to control can also include a difference in the rate of change of the level of one or more markers obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of a biomarker in a sample (e.g., benign prostatic hyperplasia or prostate cancer sample) versus a control or healthy sample, wherein the increase is above some threshold value, or a decrease in the detected level of a biomarker in a sample (e.g., benign prostatic hyperplasia or prostate cancer sample) versus a control or healthy sample, wherein the decrease is below some threshold value. The threshold value can be determine by any suitable means by measuring the biomarker levels in a plurality of tissues or samples known to have abnormal prostate state, e.g., LUTS, BPH, or prostate cancer, and comparing those levels to a normal or control sample and calculating a statistically significant threshold value.

The term “control level” or “normal level” refers to an accepted or pre-determined level of a marker in a subject sample. In some embodiments, a control level can be a range of values. In some embodiments, marker levels can be compared to a single control value, to a range of control values, to the upper level of normal, or to the lower level of normal as appropriate for the assay.

In some embodiments, the control is a standardized control, such as, for example, a control which is predetermined using an average of the levels of expression of one or more markers from a population of subjects with a normal prostate, especially subjects having no BPH or prostate cancer. In some embodiments, a control level of a marker is the level of the marker in a non-prostatic sample derived from the subject having an abnormal or enlarged prostate state.

In some embodiments, a control can be a sample from a subject at an earlier time point, e.g., a baseline level prior to suspected presence of disease, before the diagnosis of a disease, at an earlier assessment time point during watchful waiting, before treatment with a specific agent (e.g., chemotherapy, hormone therapy) or intervention (e.g., radiation, surgery). In certain embodiments, a change in the level of the marker in a subject can be more significant than the absolute level of a marker, e.g., as compared to control.

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least about four weeks earlier. In certain embodiments, an earlier time point is at least about six weeks earlier. In certain embodiments, an earlier time point is at least about two months earlier. In certain embodiments, an earlier time point is at least about three months earlier. In certain embodiments, an earlier time point is at least about six months earlier. In certain embodiments, an earlier time point is at least about nine months earlier. In certain embodiments, an earlier time point is at least about one year earlier. Multiple subject samples (e.g., about 3, about 4, about 5, about 6, about 7, or more) can be obtained at regular or irregular intervals over time and analyzed for trends in changes in marker levels. Appropriate intervals for testing for a particular subject can be determined by one of skill in the art based on ordinary considerations.

As used herein, the terms “biopsy”, as used herein with reference to a prostate biopsy, means a cell sample, collection of cells, or bodily fluid removed from a subject or patient for analysis. In some embodiments, the biopsy is a punch biopsy, endoscopic biopsy, needle biopsy, shave biopsy, incisional biopsy, excisional biopsy, or surgical resection.

As used herein, the terms “bodily fluid” means any fluid from isolated from a subject including, but not necessarily limited to, blood sample, serum sample, plasma sample, urine sample, mucus sample, saliva sample, and sweat sample. The sample may be obtained from a subject by any means such as intravenous puncture, biopsy, swab, capillary draw, lancet, needle aspiration, collection by simple capture of excreted fluid.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

As used herein, the term “subject,” “individual” or “patient,” used interchangeably, means any animal, including mammals, such as mice, rats, other rodents, rabbits, dogs, cats, swine, cattle, sheep, horses, or primates, such as humans. Tissues, cells and their progeny obtained in vivo or cultured in vitro are also encompassed by the definition of the term subject.

The term “subject” is used throughout the specification to describe an animal from which a sample is taken. In some embodiments, the subject is a human. For diagnosis of those conditions which are specific for a specific subject, such as a human being, the term “patient” may be interchangeably used. In some embodiments, the subject may be a human suspected of having or being identified as at risk to develop an abnormal prostate state, e.g., BPH, LUTS< or prostate cancer. In some embodiments, the subject may be a human suspected of having or being identified as at risk to develop prostate cancer. In some embodiments, the subject may be a human suspected of having or being identified as at risk to develop a benign condition, e.g., LUTS or BPH. In some embodiments, the subject may be a human subject with an elevated PSA level. In other embodiments, the subject may be a human subject with a normal PSA level. In other embodiments, the subject may be a human subject with an enlarged prostate, e.g., as determined by a DRE. In other embodiment, the subject may be a human subject without an enlarged prostate, e.g., as determined by a DRE. In other embodiments, the human subject may have undergone one or more prostate biopsies. In some embodiments, the subject may be diagnosed as having a resistance to one or a plurality of treatments to treat a disease or disorder afflicting the subject. In some embodiments, the subject is suspected of having or has been diagnosed with prostate cancer having a Gleason score of between 5 and 7. In some embodiments, the subject is suspected of having or has been diagnosed with prostate cancer having a Gleason score of 5, 6, or 7. In some embodiments, the subject is suspected of having or has been diagnosed with prostate cancer having a Gleason score of 8 or greater. In some embodiments, the subject is suspected of having or has been diagnosed with prostate cancer having a Gleason score of 7, 8, 9, or 10. In some embodiments, the subject may be a human suspected of having or being identified as at risk to a terminal condition or disorder. In some embodiments, the subject may be a mammal which functions as a source of the isolated sample of biopsy or bodily fluid. In some embodiments, the subject may be a non-human animal from which a sample of biopsy or bodily fluid is isolated or provided.

As used herein, the phrase “subject suspected of having or being at risk for having an abnormal prostate state” refers to a subject that presents one or more symptoms indicative of an abnormal prostate state, e.g., inflammation, LUTS, BPH or prostate cancer, or is being screened for an abnormal prostate state (e.g., during a routine physical, by PSA test, or by digital rectal examination (DRE). A subject suspected of having an abnormal prostate state may also have one or more risk factors. A subject suspected of having an abnormal prostate state has generally not been tested for cancer. However, a “subject suspected of having an abnormal prostate state” encompasses an individual who has received an initial diagnosis (e.g., a CT scan showing a mass, an enlarged prostate as determined by digital rectal examination (DRE), increased prostate inflammation, or an increased PSA level, e.g., a prostate level between about 4-10 ng/mL) based on a screening test, but for whom the type of abnormal prostate state is not known. The term further includes people who once had cancer (e.g., an individual in remission).

The terms “disorders”, “diseases”, and “abnormal state” are used inclusively and refer to any deviation from the normal structure or function of any part, organ, or system of the body, or any combination thereof. In some embodiments, a specific disease is manifested by characteristic symptoms and signs, including biological, chemical, and physical changes, and is often associated with a variety of other factors including, but not limited to, demographic, environmental, employment, genetic, and medically historical factors. In some embodiments, certain characteristic signs, symptoms, and related factors can be quantitated through a variety of methods to yield important diagnostic information. In some embodiments, the disorder, disease, or abnormal state is an abnormal prostate state, including LUTS, BPH, or prostate cancer. The abnormal prostate state of prostate cancer can be further subdivided into stages and grades of prostate cancer as provided, for example in Prostate. In: Edge S B, Byrd D R, Compton C C, et al., eds.: AJCC Cancer Staging Manual. 7th ed. New York, NY: Springer, 2010, pp 457-68 (incorporated herein by reference in its entirety). Further, abnormal prostate states can be classified as one or more of benign prostate hyperplasia (BPH), including LUTS and inflammation.

“Therapeutically effective amount” or “effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., reduce prostate volume, reduce lower urinary tract symptoms (LUTS), treat BPH, prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

As used herein, the terms “treat,” “treated,” or “treating” can refer to therapeutic treatment and/or prophylactic or preventative measures wherein the object is to prevent or slow down (lessen) an undesired physiological condition, disorder or disease, e.g., BPH or prostate cancer, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment. Exemplary treatment for BPH include, but are not limited to, a selective α₁-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

As used herein, the terms “diagnose,” “diagnosing,” or variants thereof refer to identifying the nature of a physiological condition, disorder or disease. In some embodiments, diagnosing refers to diagnosing benign prostatic hyperplasia (BPH). In some embodiments, diagnosing refers to distinguishing or differentiating between benign prostatic hyperplasia (BPH) and prostate cancer (PCa). In some embodiments, diagnosing a subject refers to identifying whether a condition is benign, pre-malignant, or malignant. In some embodiments, the condition is derived from or in the prostate of the subject. In some embodiments, diagnosing refers to determining the level of FLNA in a sample, alone or in combination with determining the age and/or prostate volume of the subject from whom the sample was derived.

As used herein, “benign” is to be contrasted with “malignant”. The terms “benign” and “malignant” are intended to convey their ordinary meaning. Therefore, “malignant” when modifying a growth is intended to refer to an abnormal growth or hyperproliferative state that is characterized by invasive or potentially invasive growth causing destruction of local tissues and cells, often leading to metastasis and death in the absence of treatment. In contrast, “benign” is intended to refer to an abnormal growth state wherein the growth does not result in the invasion of the local tissue, metastasis, or death. As used herein, “pre-malignant” is intended to refer to an abnormal growth state of a cell or group of cells prior to the biochemical alterations that cause the cell or group of cells to become malignant.

As used herein, “benign prostatic hyperplasia” or “BPH” refers to a non-cancerous (benign) enlargement of the prostate gland. Benign prostatic hyperplasia is characterized by smooth muscle and epithelial proliferation primarily within the prostatic transition zone that frequently causes lower urinary tract symptoms (LUTS) (Affenberg, et al., Urol Clin North Am. 2009 November; 36(4):443-59). “Lower urinary tract symptoms” or “LUTS”, as used herein, include, but are not limited to, difficulty urinating, impact on frequency and urgency of urination, weak force of urination stream, terminal dribbling, urinary tract infections, and erectile dysfunction. As used herein, the term “BPH” includes lower urinary tract symptoms, also referred to herein as LUTS.

As used herein, “prostate cancer,” refers to any malignant or pre-malignant form of cancer of the prostate. The term includes prostate in situ carcinomas, invasive carcinomas, metastatic carcinomas and pre-malignant conditions. The term also encompasses any stage or grade of cancer in the prostate. Where the prostate cancer is “metastatic,” the cancer has spread or metastasized beyond the prostate gland to a distant site, such as a lymph node or to the bone. The term prostate cancer does not include benign prostatic hyperplasia (BPH).

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “staging” refers to commonly used systems for grading/stating cancer, e.g., prostate cancer. In one aspect, staging can take the form of the “Gleason Score”, as well known in the art, is the most commonly used system for the grading/staging and prognosis of adenocarcinoma. The system describes a score between 2 and 10, with 2 being the least aggressive and 10 being the most aggressive. The score is the sum of the two most common patterns (grade 1-5) of tumor growth found. To be counted a pattern (grade) needs to occupy more than 5% of the biopsy specimen. The scoring system requires biopsy material (core biopsy or operative specimens) in order to be accurate; cytological preparations cannot be used. The “Gleason Grade” is the most commonly used prostate cancer grading system. It involves assigning numbers to cancerous prostate tissue, ranging from 1 through 5, based on how much the arrangement of the cancer cells mimics the way normal prostate cells form glands. Two grades are assigned to the most common patterns of cells that appear; these two grades (they can be the same or different) are then added together to determine the Gleason score (a number from 1 to 10). A Gleason score of between 5 and 7 is considered herein to be an intermediate Gleason score. A Gleason score of 8 or above is considered herein to be a high Gleason score, and refers to aggressive prostate cancer.

The Gleason system is based exclusively on the architectural pattern of the glands of the prostate tumor. It evaluates how effectively the cells of any particular cancer are able to structure themselves into glands resembling those of the normal prostate. The ability of a tumor to mimic normal gland architecture is called its differentiation, and experience has shown that a tumor whose structure is nearly normal (well differentiated) will probably have a biological behavior relatively close to normal, i.e., that is not very aggressively malignant.

A Gleason grading from very well differentiated (grade 1) to very poorly differentiated (grade 5) is usually done for the most part by viewing the low magnification microscopic image of the cancer. There are important additional details which require higher magnification, and an ability to accurately grade any tumor is achieved only through much training and experience in pathology. Gleason grades 1 and 2: These two grades closely resemble normal prostate. They are the least important grades because they seldom occur in the general population and because they confer a prognostic benefit which is only slightly better than grade 3. Both of these grades are composed by mass; in grade 2 they are more loosely aggregated, and some glands wander (invade) into the surrounding muscle (stroma). Gleason grade 3 is the most common grade and is also considered well differentiated (like grades 1 and 2). This is because all three grades have a normal “gland unit” like that of a normal prostate; that is, every cell is part of a circular row which forms the lining of a central space (the lumen). The lumen contains prostatic secretion like normal prostate, and each gland unit is surrounded by prostate muscle which keeps the gland units apart. In contrast to grade 2, wandering of glands (invading) into the stroma (muscle) is very prominent and is the main defining feature. The cells are dark rather than pale and the glands often have more variable shapes.

Gleason Grade 4 is probably the most important grade because it is fairly common and because of the fact that if a lot of it is present, patient prognosis is usually (but not always) worsened by a considerable degree. Grade 4 also shows a considerable loss of architecture. For the first time, disruption and loss of the normal gland unit is observed. In fact, grade 4 is identified almost entirely by loss of the ability to form individual, separate gland units, each with its separate lumen (secretory space). This important distinction is simple in concept but complex in practice. The reason is that there are a variety of different-appearing ways in which the cancer's effort to form gland units can be distorted. Each cancer has its own partial set of tools with which it builds part of the normal structure. Grade 4 is like the branches of a large tree, reaching in a number of directions from the (well differentiated) trunk of grades 1, 2, and 3. Much experience is required for this diagnosis, and not all patterns are easily distinguished from grade 3. This is the main class of poorly differentiated prostate cancer, and its distinction from grade 3 is the most commonly important grading decision.

Gleason grade 5 is an important grade because it usually predicts another significant step towards poor prognosis. Its overall importance for the general population is reduced by the fact that it is less common than grade 4, and it is seldom seen in men whose prostate cancer is diagnosed early in its development. This grade too shows a variety of patterns, all of which demonstrate no evidence of any attempt to form gland units. This grade is often called undifferentiated, because its features are not significantly distinguishing to make it look any different from undifferentiated cancers which occur in other organs. When a pathologist looks at prostate cancer specimens under the microscope and gives them a Gleason grade, an attempt to identify two architectural patterns and assign a Gleason grade to each one is made. There may be a primary or most common pattern and then a secondary or second most common pattern which the pathologist will seek to describe for each specimen; alternatively, there may often be only a single pure grade. In developing his system, Dr. Gleason discovered that by giving a combination of the grades of the two most common patterns he could see in any particular patient's specimens, that he was better able to predict the likelihood that a particular patient would do well or badly. Therefore, although it may seem confusing, the Gleason score which a physician usually gives to a patient, is actually a combination or sum of two numbers which is accurate enough to be very widely used. These combined Gleason sums or scores may be determined as follows:

The lowest possible Gleason score is 2 (1+1), where both the primary and secondary patterns have a Gleason grade of 1 and therefore when added together their combined sum is 2. Very typical Gleason scores might be 5 (2+3), where the primary pattern has a Gleason grade of 2 and the secondary pattern has a grade of 3, or 6 (3+3), a pure pattern. Another typical Gleason score might be 7 (4+3), where the primary pattern has a Gleason grade of 4 and the secondary pattern has a grade of 3. Finally, the highest possible Gleason score is 10 (5+5), when the primary and secondary patterns both have the most disordered Gleason grades of 5.

Another way of staging prostate cancer is by using the TNM System. It describes the extent of the primary tumor (T stage), the absence or presence of spread to nearby lymph nodes (N stage) and the absence or presence of distant spread, or metastasis (M stage). Each category of the TNM classification is divided into subcategories representative of its particular state. For example, primary tumors (T stage) may be classified into:
T1: The tumor cannot be felt during a digital rectal exam, or seen by imaging studies, but cancer cells are found in a biopsy specimen;
T2: The tumor can be felt during a DRE and the cancer is confined within the prostate gland;
T3: The tumor has extended through the prostatic capsule (a layer of fibrous tissue surrounding the prostate gland) and/or to the seminal vesicles (two small sacs next to the prostate that store semen), but no other organs are affected;
T4: The tumor has spread or attached to tissues next to the prostate (other than the seminal vesicles).

Lymph node involvement is divided into the following 4 categories:
NO: Cancer has not spread to any lymph nodes;
N1: Cancer has spread to a single regional lymph node (inside the pelvis) and is not larger than 2 centimeters;
N2: Cancer has spread to one or more regional lymph nodes and is larger than 2 centimeters, but not larger than 5 centimeters; and
N3: Cancer has spread to a lymph node and is larger than 5 centimeters (2 inches). Metastasis is generally divided into the following two categories:
MO: The cancer has not metastasized (spread) beyond the regional lymph nodes; and
Ml: The cancer has metastasized to distant lymph nodes (outside of the pelvis), bones, or other distant organs such as lungs, liver, or brain.

In addition, the Tstage is further divided into subcategories T1a-c T2a-c, T3a-c and T4a-b. The characteristics of each of these subcategories are well known in the art and can be found in a number of textbooks.

As used herein, the term “biomarker” is understood to mean a measurable characteristic that reflects in a quantitative or qualitative manner the physiological state of an organism. The physiological state of an organism is inclusive of any disease or non-disease state, e.g., a subject having an abnormal prostate state such as LUTS, BPH, or prostate cancer or a subject who is otherwise healthy. Said another way, biomarkers are characteristics that can be objectively measured and evaluated as indicators of normal processes, pathogenic processes, or pharmacologic responses to a therapeutic intervention. Biomarkers can be clinical parameters (e.g., age), laboratory measures (e.g., molecular biomarkers, such as prostate specific antigen (PSA) and FLNA), genetic or other molecular determinants, such as phosphorylation or acetylation state of a protein marker, methylation state of nucleic acid, clinical biomarkers, such as imaging-based measures, e.g., prostate volume, and age, or any other detectable molecular modification to a biological molecule. In some embodiments, examples of biomarkers include, for example, polypeptides, peptides, polypeptide fragments, proteins, antibodies, hormones, polynucleotides, RNA or RNA fragments, microRNA (miRNAs), lipids, polysaccharides, and other bodily metabolites. In some embodiments, other examples of biomarkers include the age of the subject and the volume of the prostate.

In some embodiments, a biomarker of the present invention (e.g., FLNA) is modulated (e.g., increased or decreased level) in a biological sample from a subject or a group of subjects having a first phenotype (e.g., having a disease) as compared to a biological sample from a subject or group of subjects having a second phenotype (e.g., not having the disease, e.g., a control). A biomarker may be differentially present at any level, but is generally present at a level that is increased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). In some embodiments, a biomarker is differentially present at a level that is statistically significant (e.g., a p-value less than 0.05 and/or a q-value of less than 0.10 as determined using either Welch's T-test or Wilcoxon's rank-sum Test).

As used herein, “detecting”, “detection”, “determining”, and the like are understood to refer to an assay performed for identification of one or more specific markers in a sample. In some embodiments, detecting refers to an assay to identify the presence, absence, or quantity of FLNA in a sample. In some embodiments, detecting refers to an assay to identify the presence of an enlarged prostate. In some embodiments, detecting refers to an assay to identify the prostate volume. In some embodiments, detecting may include identifying the presence, absence, or quantity of an additional one or more specific markers in a sample, e.g., filamin A (FLNA), alone or in combination with one of more additional prostate cancer markers, e.g., one or more (e.g., 1, 2, 3, 4, 5, 6, 7, 8, or 9 or more) markers selected from the group consisting of, for example, PSA, filamin B, LY9, keratin 4, keratin 7, keratin 8, keratin 15, keratin 18, keratin 19, tubulin-beta 3, PSM, PSCA, TMPRSS2, PDEF, HPG-1, PCA3, and PCGEM1. In some embodiments, the amount of marker expression or activity detected in the sample can be none or below the level of detection of the assay or method. In some embodiments, the level of protein biomarker is detected in a sample. In a preferred embodiment an IPMRM assay is used to detect the level of a protein biomarker in a sample. IPMRM assays for detecting FLNA are described herein, and are also described in, for example, U.S. patent application Ser. No. 15/801,093, filed on Nov. 1, 2017, the contents of which are hereby incorporated herein by reference.

In certain embodiments, differentiating benign prostatic hyperplasia (BPH) from prostate cancer in a subject is carried out by detecting the protein level of Filamin A (FLNA) in a biological sample from the subject; measuring the prostate volume of the subject; analyzing the age of the subject, the prostate volume of the subject, and the protein level of FLNA in the biological sample with corresponding predetermined threshold value for FLNA level, age and prostate volume; and determining whether the subject has BPH or prostate cancer by comparing the age of the subject, the prostate volume of the subject, and the protein level of FLNA in the biological sample to the corresponding threshold value. If the protein level of FLNA, prostate volume, and age of the subject is altered from the predetermined threshold value, the subject is identified as having either BPH or prostate cancer. For example, in some embodiments, if the level of FLNA, prostate volume, and age of the subject is below corresponding predetermined threshold value, the subject is diagnosed with BPH, and if the protein level of FLNA, prostate volume, and age of the subject is above the corresponding predetermined threshold value, the subject is diagnosed with prostate cancer.

In other embodiments, diagnosis or monitoring BPH or prostate cancer in a subject is carried out by detecting the protein level of Filamin A (FLNA) in a biological sample from the subject; measuring the prostate volume of the subject; analyzing the age of the subject, the prostate volume of the subject, and the protein level of FLNA in the biological sample with corresponding predetermined threshold value for FLNA level, age and prostate volume; and determining whether the subject has BPH or prostate cancer by comparing the age of the subject, the prostate volume of the subject, and the protein level of FLNA in the biological sample to the corresponding threshold value. If the protein level of FLNA, prostate volume, and age of the subject are above or below the corresponding predetermined threshold value, then a diagnosis of prostate cancer or benign prostatic hyperplasia (BPH), respectively, in the subject is indicated.

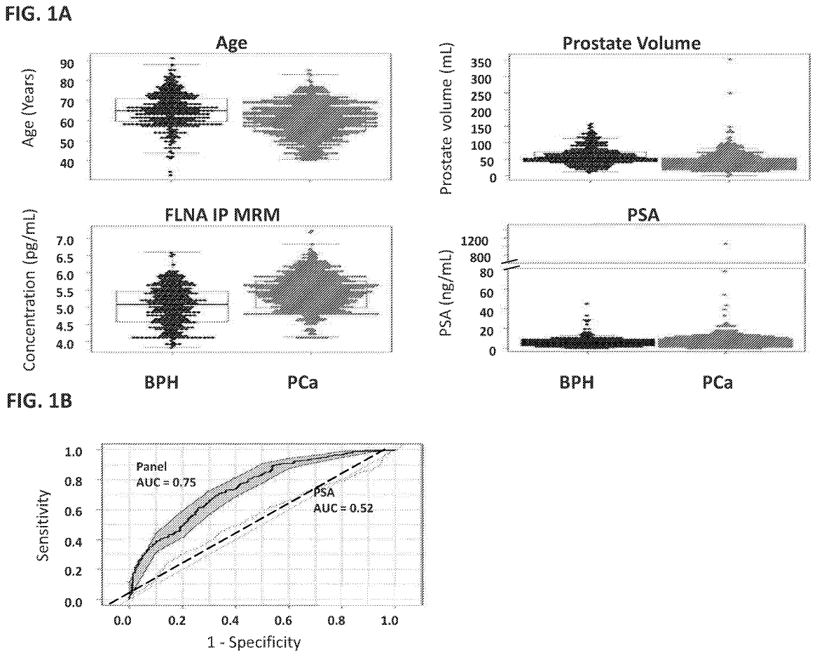

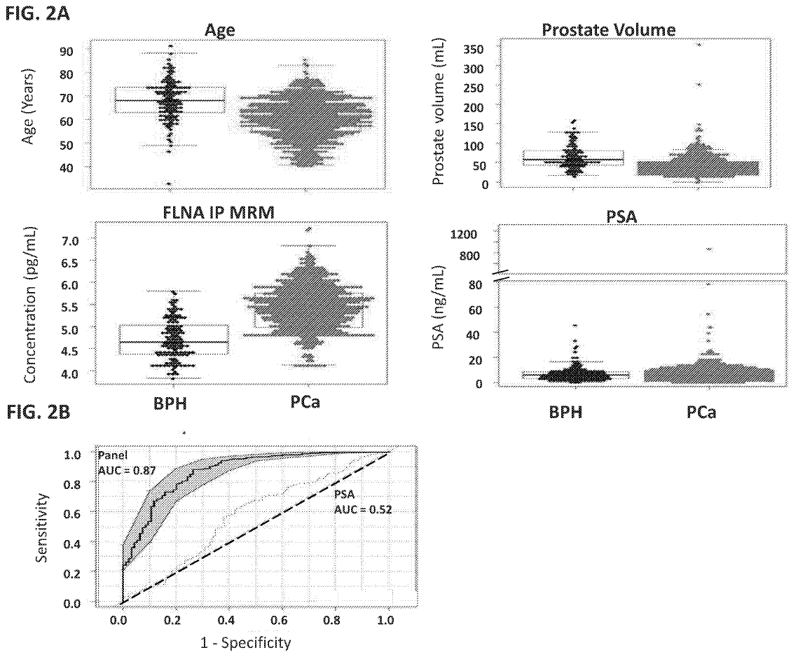

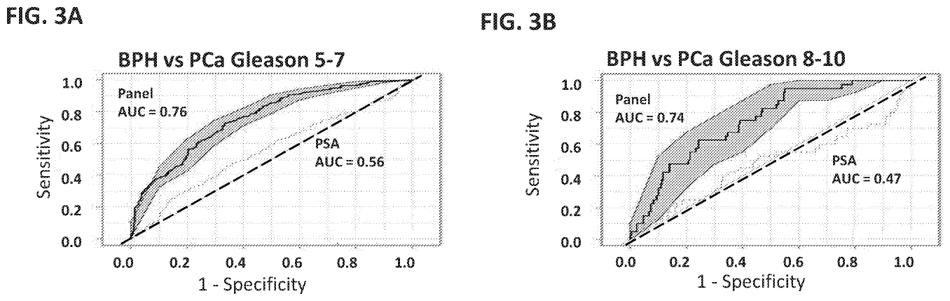

In some embodiments, the differentiating, diagnosis or monitoring can be determined based on an algorithm or computer program that predicts whether the biological sample is cancerous or benign based on the level of FLNA, the prostate volume, and age of the subject. In some embodiments, the diagnosis can be determined based on the area under the curve or AUC of the biomarkers (e.g., FLNA level, prostate volume, and age of the subject), see, e.g., FIG. 1B, FIG. 2B, FIG. 3A and FIG. 3B).

In accordance with various embodiments, algorithms may be employed to predict whether or not a biological sample is likely to be diseased, e.g., have BPH or prostate cancer, or distinguish between diseased states, e.g., distinguish between BPH and prostate cancer. The skilled artisan will appreciate that an algorithm can be any computation, formula, statistical survey, nomogram, look-up table, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output,” e.g., differentiation between BPH and prostate cancer. Any suitable algorithm, whether computer-based or manual-based (e.g., look-up table), is contemplated herein. In certain embodiments, an algorithm of the invention used to predict whether a biological sample has BPH producing a score on the basis of the detected level of FLNA in the sample in combination with age and/or prostate volume, and optionally at least one, or two, or three, or four, or five, or six, or seven, or eight, or nine or more additional prostate cancer markers (e.g., prostate specific antigen (PSA), filamin B, LY9, keratin 4, keratin 7, keratin 8, keratin 15, keratin 18, keratin 19, tubulin-beta 3, PSM, PSCA, TMPRSS2, PDEF, HPG-1, PC A3, PCGEM1, or combinations thereof). For example, wherein if the score is below a certain threshold score, then the subject has BPH, and wherein if the score is above a certain threshold score, then the subject has prostate cancer. In certain embodiments, the algorithm also produces a score using the patient's age as a continuous predictor variable.

In certain embodiments, the biomarkers of the invention can include variant sequences. More particularly, the binding agents/reagents used for detecting the biomarkers of the invention can bind and/or identify variants of the biomarkers of the invention. As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

As used herein, the term “area under the curve” or “AUC” refers to the area under the curve in a plot of sensitivity versus specificity. For example, see FIGS. 1B, 2B and 3A-3B. In some embodiments, the AUC for a biomarker, or combination of biomarkers, is at least about 0.5. In some embodiments, the AUC for a biomarker, or combination of biomarkers, is at least about 0.6. In some embodiments, the AUC for a biomarker, or combination of biomarkers, is at least about 0.7. In some embodiments, the AUC for a biomarker, or combination of biomarkers, of the invention is at least about 0.8. In some embodiments, the AUC for a biomarker, or combination of biomarkers, of the invention is at least about 0.9. In some embodiments, the AUC for a biomarker, or combination of biomarkers, of the invention is at least about 1.0. In some embodiments, the AUC for a biomarker, or combination of biomarkers, of the invention is 0.5, 0.51, 0.52, 0.53, 0.54, 0.55, 0.56, 0.57, 0.58, 0.59, 0.6, 0.61, 0.62, 0.63, 0.64, 3.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 or 1.0. In some embodiments, the AUC for a biomarker, or combination of biomarkers, of the invention is at least 0.5, 0.51, 0.52, 0.53, 0.54, 0.55, 0.56, 0.57, 0.58, 0.59, 0.6, 0.61, 0.62, 0.63, 0.64, 3.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 or 1.0. In one embodiment, the combination of biomarkers comprises FLNA level, age, and prostate volume.

As used herein, a “predetermined threshold value” or “threshold value” of a biomarker or panel of biomarkers refers to the level of the biomarker(s) (e.g., the expression level or quantity (e.g., ng/ml) of FLNA in a biological sample), in a corresponding control/normal sample or group of control/normal samples obtained from one or more subjects, e.g., normal or healthy subjects, e.g., those males that do not have an abnormal prostate state, or subjects with an abnormal prostate state, as well as age and/or prostate volume. The predetermined threshold value may be determined prior to or concurrently with measurement of marker levels, e.g., FLNA marker levels, in the biological sample, age, and/or prostate volume. The control sample may be from the same subject at a previous time or from different subjects.

Without any particular limitation, a method according to the present invention may involve discerning whether a subject has BPH or prostate cancer comprising obtaining a first series of biological samples from a first group of patients predetermined to be suffering from one abnormal prostate state, and a second series of biological samples from a second group of patients predetermined not to be suffering from the same abnormal prostate state. The second group of patients may suffer from a different abnormal prostate state than the first group, or alternatively not suffer from any abnormal prostate state. A threshold value for discerning between the first and second patient groups may be generated by detecting one or more biomarkers (including FLNA, prostate volume and age) in the first and second series of biological samples to thereby obtain a biomarker level for each biomarker in each biological sample of each series. The levels may be combined in a manner that allows discrimination between samples from the first and second group of patients. A threshold value may be selected from the combined levels in a suitable manner such as any one or more of a method that: reduces the misclassification rate between the first and second group of patients; increases or maximizes the sensitivity in discriminating between the first and second group of patients; and/or increases or maximizes the specificity in discriminating between the first and second group of patients. The threshold value can be used as a basis to discriminate between the presence and absence of the abnormal prostate state that the first group of patients suffered from in a given candidate sample. Hence, a biological sample from a subject who's status in relation to the abnormal prostate state is undetermined may be obtained and the same biomarker/s that served as the basis for generating the threshold value measured in the same manner as for the first and second patient groups to obtain a patient biomarker value. The patient biomarker value derived from the quantified biomarker level/s can then be compared to the threshold value for a determination of the abnormal prostate state to be made. A suitable algorithm and/or transformation of individual or combined biomarker levels obtained from the subject's biological sample may be used to generate the patient biomarker value for comparison to the threshold value. In some embodiments, one or more parameters used in deriving the threshold value and/or the patient biomarker value are weighted.

In some embodiments, the patient receives a negative diagnosis for the abnormal prostate state if the patient biomarker value is less than the threshold value. In some embodiments, the patient receives a negative diagnosis for the abnormal prostate if the patient biomarker value is more than the threshold value. In some embodiments, the patient receives a positive diagnosis for the abnormal prostate if the patient biomarker value is less than the threshold value. In some embodiments, the patient receives a positive diagnosis for the abnormal prostate if the patient biomarker value is more than the threshold value. In some embodiments, the abnormal prostate is a non-cancerous prostate disease (e.g. BPH). In some embodiments, the abnormal prostate is a prostate cancer.

One non-limiting example for conducting these analyses is Receiver Operating Characteristic (ROC) curve analysis. Generally, the ROC analysis may involve comparing a classification for each patient tested to a ‘true’ classification based on an appropriate reference standard. Classification of multiple patients in this manner may allow derivation of measures of sensitivity and specificity. Sensitivity will generally be the proportion of correctly classified patients among all of those that are truly positive, and specificity the proportion of correctly classified cases among all of those that are truly negative. In general, a trade-off may exist between sensitivity and specificity depending on the threshold value selected for determining a positive classification. A low threshold may generally have a high sensitivity but relatively low specificity. In contrast, a high threshold may generally have a low sensitivity but a relatively high specificity. A ROC curve may be generated by inverting a plot of sensitivity versus specificity horizontally. The resulting inverted horizontal axis is the false positive fraction, which is equal to the specificity subtracted from 1. The area under the ROC curve (AUC) may be interpreted as the average sensitivity over the entire range of possible specificities, or the average specificity over the entire range of possible sensitivities. The AUC represents an overall accuracy measure and also represents an accuracy measure covering all possible interpretation thresholds.

The term “correlate” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

As used herein, “specific for” or “specifically binds to” means that the binding affinity of a substrate to a specified target nucleic acid or amino acid sequence, is statistically higher than the binding affinity of the same substrate to a generally comparable, but non-target nucleic acid or amino acid sequence. Normally, the binding affinity of a substrate to a specified target nucleic acid or amino acid sequence is at least 1.5 fold, and preferably 2 fold or 5 fold, of the binding affinity of the same substrate to a non-target nucleic acid or amino acid sequence. It also refers to binding of a substrate to a specified nucleic acid or amino acid target sequence to a detectably greater degree, e.g., at least 1.5-fold over background, than its binding to non-target nucleic acid or amino acid sequences and to the substantial exclusion of non-target nucleic acids or amino acids. The substrate's Kd to each nucleotide or amino acid sequence can be compared to assess the binding specificity of the substrate to a particular target nucleotide or amino acid sequence. The terms “specific binding”, “specifically binds” or “specifically binding”, as used herein in the context of an antibody, refer to non-covalent or covalent preferential binding of an antibody to an antigen relative to other molecules or moieties (e.g., an antibody specifically binds to a particular antigen relative to other available antigens). In some embodiments, an antibody specifically binds to an antigen (e.g., a tumor or viral antigen) if it binds with a dissociation constant Kp of from about 1 pM to about 500 mM. In some embodiments, the antibody or antigen binding protein has a dissociation constant Kp in a range from about 1 pM to about 1000 nM. In some embodiments, the antibody or antigen binding protein has a dissociation constant Kp in a range from about 1 pM to about 500 nM. In some embodiments, the antibody or antigen binding protein has a dissociation constant Kp in a range from about 1 pM to about 250 nM. In some embodiments, the antibody or antigen binding protein has a dissociation constant Kp in a range from about 1 pM to about 100 nM. In some embodiments, the antibody or antigen binding protein has a dissociation constant K_{D} in a range from about 1 pM to about 10 nM. In some embodiments, the antibody or antigen binding protein has a dissociation constant Kp in a range from about 1 pM to about 1 nM. In some embodiments, the antibody or antigen binding protein has a dissociation constant Kp in a range from about 1 pM to about 750 pM. In some embodiments, the antibody or antigen binding protein has a dissociation constant Kp in a range from about 1 pM to about 500 pM. In some embodiments, the antibody or binding protein has a dissociation constant Kp in a range from about 1 nM to about 100 nM.

The terms “level of expression of a gene”, “gene expression level”, “level of a marker”, and the like refer to the level of mRNA, as well as pre-mRNA nascent transcript(s), transcript processing intermediates, mature mRNA(s) and degradation products, or the level of protein, encoded by the gene in the cell. The “level” of one of more biomarkers means the absolute or relative amount or concentration of the biomarker in the sample.

A “higher level of expression”, “higher level”, and the like of a marker refers to an expression level in a test sample that is greater than the standard error of the assay employed to assess expression, and is preferably at least 10%, 15%, 20%, 25%, 30%, 35%, 40%, 45%, 50%, 55%, 60%, 65%, 70%, 75%, 80%, 85%, 90%, more than the expression level of the marker in a control sample (e.g., sample from a healthy subject not having the marker associated disease, i.e., an abnormal prostate state) and preferably, the average expression level of the marker or markers in several control samples.

A “lower level of expression” or “lower level” and the like of a marker refers to an expression level in a test sample that is less than the standard error of the assay employed to assess expression, and is preferably at least 90%, 85%, 80%, 75%, 70%, 65%, 60%, 55%, 50%, 45%, 40%, 35%, 30%, 25%, 20%, 15%, or 10% of the expression level of the marker in a control sample (e.g., sample from a healthy subjects not having the marker associated disease, i.e., an abnormal prostate state) and preferably, the average expression level of the marker in several control samples.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e., ribonucleotides, containing a non-naturally occurring nucleobase instead of a naturally occurring nucleobase such as uridines or cytidines modified at the 5-position, e.g., 5-(2-amino) propyl uridine, 5-bromo uridine; adenosines and guanosines modified at the 8-position, e.g. 8-bromo guanosine; deaza nucleotides, e.g., 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N2 or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

As used herein, a “probe” is meant to include a nucleic acid oligomer or oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

The terms “amino acid” refer to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs including non-natural analogs.

As used herein, the terms “peptide,” “polypeptide” and “protein” are used interchangeably and refer to two or more amino acids covalently linked by an amide bond or non-amide equivalent. The peptides of the disclosure can be of any length. For example, the peptides can have from about two to about 100 or more residues, such as, 5 to 12, 12 to 15, 15 to 18, 18 to 25, 25 to 50, 50 to 75, 75 to 100, or more in length. Preferably, peptides are from about 2 to about 18 residues in length. The peptides of the disclosure also include l- and d-isomers, and combinations of l- and d-isomers. The peptides can include modifications typically associated with posttranslational processing of proteins, for example, cyclization (e.g., disulfide or amide bond), phosphorylation, glycosylation, carboxylation, ubiquitination, myristylation, or lipidation.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence upon which the sequence is derived.

As used herein, percent “homology,” “identity” or “sequence identity” in the context of two or more nucleic or amino acids, as used herein, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic or amino acids of the invention are substantially identical, meaning they have at least about 70%, at least about 75%, at least about 80%, at least about 85%, at least about 90%, and in some embodiments at least about 95%, 96%, 97%, 98%, 99% nucleotide or amino acid residue sequence identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acids, at least about 60-80 nucleotides or amino acids, or any integral value therebetween. In some embodiments, identity exists over a longer region than 60-80 nucleotides or amino acids, such as at least about 80-100 nucleotides or amino acids, and in some embodiments the sequences are substantially identical over the full length of the sequences being compared. In some embodiments, identity is determined by using the stand-alone executable BLAST engine program for blasting two sequences (blZseq), which can be retrieved from the National Center for Biotechnology Information (NCBI) ftp site, using the default parameters (Tatusova and Madden, FEMS Microbiol Lett, 1999, 174, 247-250; which is incorporated herein by reference in its entirety).

An “antigen binding protein” is a protein comprising a portion that binds to an antigen and, optionally, a scaffold or framework portion that allows the antigen binding portion to adopt a confirmation that promotes binding of the antigen binding protein to the antigen. Examples of antigen binding proteins include antibodies, antibody fragments (e.g., an antigen binding fragment of an antibody), antibody derivatives, and antibody analogs. The antigen binding protein can comprise, for example, an alternative protein scaffold or artificial scaffold with grafted CDRs or CDR fragments or variants with substantially the same binding affinity as one or more disclosed CDR amino acid sequences. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

The term “antibody” as used herein refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. The basic antibody structural unit is a tetramer. Each tetramer is composed of two identical pairs of polypeptide chains, each pair having one “light” (about 25 kDa) and one “heavy” chain (about 50-70 kDa). Generally, the amino-terminal portion of each antibody chain includes a variable region that is primarily responsible for antigen recognition. The carboxy-terminal portion of each chain defines a constant region, e.g., responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. The term antibody may mean an antibody of classes IgG, IgM, IgA, IgD or IgE, or fragments, binding fragments or derivatives thereof, including Fab, F(ab′)2, Fd, and single chain antibodies, diabodies, bispecific antibodies, bifunctional antibodies, antigen binding proteins thereof and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

The variable regions of each heavy/light chain pair (VH/VL), respectively, form the antigen binding site. The variable regions of antibody heavy and light chains (VH/VL) exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is known in the art, including, for example, definitions as described in Kabat et al. in Sequences of Proteins of Immunological Interest, 5^{th} Ed., US Dept. of Health and Human Services, PHS, NIH, NIH Publication no. 91-3242, 1991 (herein referred to as “Kabat numbering”). For example, the CDR regions of an antibody can be determined according to Kabat numbering.

The terms “intact antibody” or “full length antibody” refer to an antibody composed of two identical antibody light chains and two identical antibody heavy chains that each contain an Fc region.

An “antigen binding domain,” “antigen binding region,” or “antigen binding site” is a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

An “epitope” is the portion of a molecule that is bound by an antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein). Generally the variable regions, particularly the CDRs, of an antibody interact with the epitope.

The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic-lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known See, for example, Bowie et al. Science 253:164 (1991), which is incorporated by reference in its entirety.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, at least about 55%, at least about 60%, at least about 70%, at least about 75%, at least about 80%, at least about 85%, at least about 90%, at least about 95%, at least about 96%, at least about 97%, at least about 98%, or at least about 99% of the nucleotide residues of the first portion are capable of base pairing with nucleotide residues in the second portion. In some embodiments, all nucleotide residues of the first portion are capable of base pairing with nucleotide residues in the second portion.

As used herein, “surrogate peptide” is understood as any peptide derived from a FLNA marker wherein the surrogate peptide is prepared by digesting the marker protein, e.g., FLNA, with a protease of known specificity (e.g., trypsin or endoproteinase Lys-C), and wherein the peptide can be used as a surrogate reporter to determine the abundance of the FLNA marker protein, and optionally isoforms or fragments thereof, in a sample, using a mass spectrometry-based assay, e.g., MRM or IPMRM. Surrogate peptides can be tryptic peptides between, for example, about 8 and 22 amino acids. Surrogate peptides can be chosen by methods known in the art, e.g., Skyline® software and LC-MS/MS analysis (LTQ Orbitrap Velos™ coupled to Eksigent nano-LC™) of recombinant protein (GenScript®) tryptic digest. Surrogate peptides can be chosen based on surrogate peptide selection rules (Halquist, et al., Biomed Chromatography 25 (1-2):47-58) and signal intensities of the peptides in spiked and unspiked serum digests. The uniqueness of the surrogate peptides to the target FLNA marker can be confirmed by BLAST searches.

As used herein, the term “kit” refers to a set of components provided in the context of a system for diagnosing or monitoring a subject for BPH or prostate cancer, or for differentiating between BPH and prostate cancer in a subject. Such delivery systems may include, for example, systems that allow for storage, transport, or delivery of various diagnostic or therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a petri dish or polysterene plate for use in a cell culture assay, while a second container may contain cells, such as control cells. As another example, the kit may comprise a first container comprising a solid support such as a chip or slide with one or a plurality of ligands with affinities to one or a plurality of biomarkers disclosed herein and a second container comprising any one or plurality of reagents necessary for the detection and/or quantification of the amount of biomarkers in a sample. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### C. Biomarkers of the Invention

The present invention based, at least in part, on the discovery that a biomarker panel comprising filamin A (FLNA) (e.g., serum FLNA concentration), in combination with one or more clinical biomarkers selected from age and prostate volume, is able to differentiate benign prostate hyperplasia (BPH), including LUTS, from prostate cancer in a subject more effectively than PSA alone. In one embodiment, the biomarker panel comprises FLNA, age, and prostate volume. In one embodiment, the subject has had one or more prostate biopsies. In another embodiment, the subject has had multiple prostate biopsies. In another embodiment, the subject has had prior screening, e.g., PSA test, DRE, or negative biopsy.

The present invention based, also in part, on the discovery that FLNA level (e.g., serum FLNA concentration), in combination with one or more clinical biomarkers selected from age and prostate volume, is able to differentiate BPH from prostate cancer in a subject, wherein the prostate cancer is characterized as having an intermediate Gleason score of between 5 and 7, an intermediate Gleason score of 5 or 6, a high Gleason score of 7, 8, 9, or 10, or a high Gleason score of 8 or above.

The ability to distinguish between BPH and prostate cancer allows for more accurate diagnosis and stratification between prostate cancer and BPH, for example wherein a subject has had prior screening, e.g., with PSA or digital rectal exam (DRE), and/or is suspected of having an abnormal prostate state such as LUTS, BPH or prostate cancer. Screening or monitoring a subject, e.g., a subject who has had prior screening and/or is suspected of having an abnormal prostate state, and/or has had a negative biopsy, using the biomarker panel described herein, provides for the differentiation between BPH and prostate cancer and therefore avoids costly and invasive unnecessary procedures such as prostate biopsy.

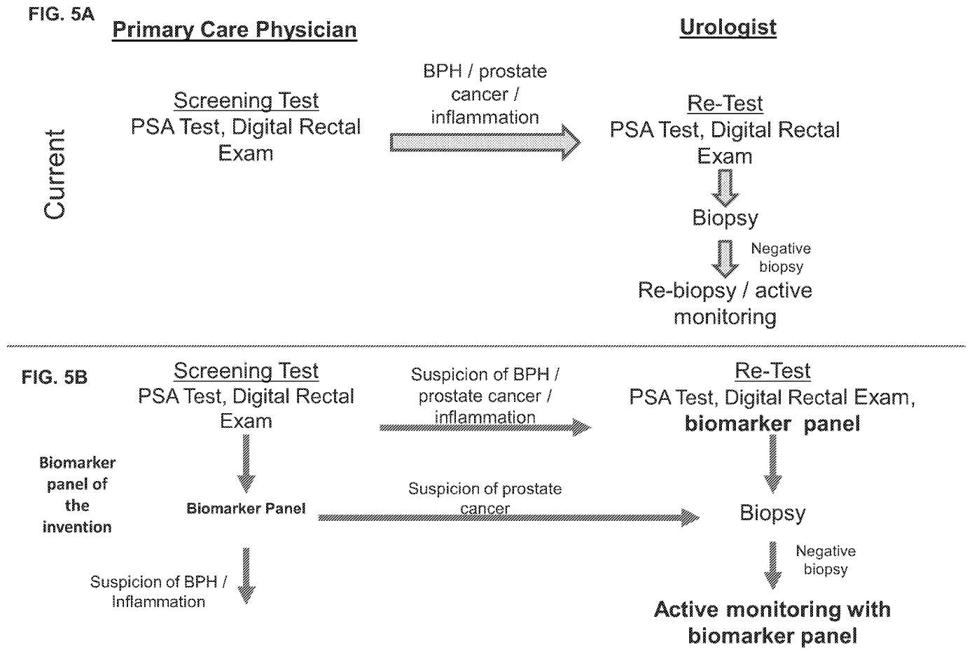

Accordingly, the invention provides methods for differentiating BPH from prostate cancer in a subject using the biomarker panel described herein. The invention also provides methods for diagnosing and/or monitoring BPH in a subject, e.g., a subject suspected of having an abnormal prostate state such as LUTS, BPH, or prostate cancer using the biomarker panel described herein (see, for example, FIG. 5B). Based on the ability to differentiate BPH from prostate cancer in a subject, the present invention also provides methods for avoiding unnecessary prostate biopsy in a subject using the biomarker panel described herein.

The invention also provides methods for treating BPH in a subject using the biomarker panel described herein.

### Filamin A

Filamin A (FLNA) is also known as FLN-A, FLN1, ABP-280, OPD1, OPD2, Endothelial Actin-Binding Protein, CVD1, FMD, MNS, NHBP, XLVD, XMVD, Actin Binding Protein 280, Alpha-Filamin, Filamin-1, and Filamin-A, each of which may appear herein and are considered equivalent terms as used herein, is a 280-kD protein that is thought to crosslink actin filaments into orthogonal networks in cortical cytoplasm. The large molecular-weight protein also participates in the anchoring of membrane proteins to the actin cytoskeleton. Remodeling of the cytoskeleton is central to the modulation of cell shape and migration cells. FLNA has previously been associated with various cancers.

Filamin A, encoded by the FLNA gene, is a widely expressed protein that regulates reorganization of the actin cytoskeleton by interacting with integrins, transmembrane receptor complexes, and second messengers. At least two different isoforms are known, isoform 1 and isoform 2, all of which are contemplated by the invention and encompassed by the term “filamin A” and/or “FLNA”. It will be appreciated that isoform 1 is the predominant transcript encoding filamin A. Isoform 2 includes an alternate in-frame exon and encodes a slightly longer protein isoform. Interaction with FLNA may allow neuroblast migration from the ventricular zone into the cortical plate. FLNA tethers cell surface-localized furin, modulates its rate of internalization and directs its intracellular trafficking. Further reference to FLNA can be found in the scientific literature, for example, in Gorlin J B et al., (October 1993). “Actin-binding protein (ABP-280) filamin gene (FLN) maps telomeric to the color vision locus (R/GCP) and centromeric to G6PD in Xq28”. Genomics 17 (2): 496-8, and Robertson S P et al. (March 2003). “Localized mutations in the gene encoding the cytoskeletal protein FLNA cause diverse malformations in humans”. Nat Genet 33 (4): 487-91, each of which are incorporated herein by reference. The nucleotide and amino acid sequences of FLNA can be found as GenBank Accession No. NM_001456.3 (FLNA, isoform 1, mRNA transcript sequence, SEQ ID NO: 31) and the corresponding polypeptide sequence of NP_001447.2 (FLNA, isoform 1, polypeptide sequence, SEQ ID NO: 32) and as GenBank Accession No. NM_001110556.1 (FLNA, isoform 2, mRNA transcript sequence, SEQ ID NO: 33) and the corresponding polypeptide sequence of NP_001104026.1 (FLNA, isoform 2, polypeptide sequence, SEQ ID NO: 34). These GenBank numbers are incorporated herein by reference in the versions available on the earliest effective filing date of this application.

The present disclosure is based, at least in part, on the discovery that a biomarker panel comprising FLNA, in combination with one or more of age and prostate volume, is able to differentiate between BPH and prostate cancer (PCa). Accordingly, the disclosure provides methods for diagnosing and monitoring BPH in a subject. The disclosure also provides methods for differentiating BPH from prostate cancer in a subject. The disclosure also provides methods for avoiding unnecessary prostate biopsy in a subject. In some embodiments, the subject has undergone one or more prostate biopsy. In other embodiments, the subject has been determined to have elevated PSA, e.g., a PSA level of 4, 5, 6, 7, 8, 9, or 10 ng/ml or greater. In another embodiment, the subject has had a digital rectal examination (DRE). In one embodiment, the subject has an enlarged prostate. In one embodiment, the subject does not have an enlarged prostate. In another embodiment, the subject has lower urinary tract symptoms (LUTS). In other embodiments, the subject has an intermediate Gleason score (e.g., a Gleason score of between 5 and 7) or a high Gleason score (e.g., a Gleason score of greater than 8). The disclosure further provides panels and kits for practicing the methods of the disclosure.

It is understood that the disclosure includes the use of any combination of one or more of the FLNA sequences provided herein or any fragments thereof as long as the fragment can allow for the specific identification of FLNA. For example, an ELISA antibody must be able to bind to the FLNA fragment so that detection is possible. Methods of the disclosure and reagents can be used to detect single isoforms of FLNA, e.g., isoform 1 and isoform 2, combinations of FLNA isoforms, or all of the FLNA isoforms simultaneously. Unless specified, FLNA can be considered to refer to one or more isoforms of FLNA, including total FLNA. Moreover, it is understood that there are naturally occurring variants of FLNA, which may or may not be associated with a specific disease state, the use of which are also included in the instant application.

Accordingly, the present disclosure also contemplates fragments and variants of FLNA. It is also understood that the disclosure encompasses the use of nucleic acid molecules encoding FLNA, including, for example, FLNA-encoding DNA, FLNA mRNA, and fragments and/or variants thereof. Reference to “FLNA” may refer to filamin A polypeptide or to the FLNA gene, unless otherwise indicated.

FLNA (and any additional biomarkers) may be detected as a polypeptide or a detectable fragment thereof. Alternatively, FLNA may be detected as a nucleic acid molecule, such as DNA, RNA, mRNA, microRNA, and the like. In addition, FLNA (and any additional biomarkers) may be detected as any combination of polypeptides and nucleic acid molecules. In certain embodiments, all of the biomarkers tested are in the form of polypeptides. In certain other embodiments, all of the biomarkers tested are in the form of polynucleotides. In certain other embodiments, at least FLNA is in the form of a polypeptide, whereas any other markers tested can be a polypeptide or nucleic acid molecule. In still other embodiments, at least FLNA is in the form of a nucleic acid molecule, whereas any other markers tested can be a polypeptide or nucleic acid molecule.

In certain embodiments, the biomarkers of the invention, e.g., FLNA, can include variant sequences. More particularly, the binding agents/reagents used for detecting the biomarkers of the invention can bind and/or identify variants of the biomarkers of the invention. As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variants of the disclosed polypeptide biomarkers are preferably themselves expressed in subjects with prostate cancer at levels that are higher or lower than the levels of expression in normal, healthy individuals.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substanti ally unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0x that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

### Age of the Subject

In some embodiments, the biomarker panel of the invention comprises age of the subject. As used herein, the term “age” refers to the length of time that a subject has been alive. For example, the age of a subject is calculated from the date of birth of the subject to the current date.

Age as a biomarker, in combination with FLNA and prostate volume, can be used as a continuous predictive variable for differentiating between benign prostatic hyperplasia (BPH) and prostate cancer (PCa). For example, increased age is associated with increased risk of both BPH and PCa. Conversely, decreased age is associated with decreased risk of BPH and PCa. In some embodiments, an age over about 50, 55, 60, 65, 70, 75, 80, or 85 years is associated with increased risk of BPH and PCa.

### Prostate Volume of the Subject

In some embodiments, the biomarker panel of the invention comprises prostate volume of the subject. Prostate volume can be measured by any means known in the art. Three major techniques are in widespread use to determine prostate size. From a transrectal ultrasound (TRUS), volume can be estimated via the traditional ellipsoid estimation based on the height (H), width (W), and length (L) of the prostate, using the formula: H×W×L×0.523. Alternatively, the same TRUS can be contoured on multiple axial slices (of thickness typically between 2.5 mm and 5 mm) during a brachytherapy volume study and then integrated in 3D space to generate a contoured volume estimate. Finally, MRI are being increasingly used to stage prostate cancer and commonly report volume estimates based on the ellipsoid formula (see Murciano-Goroff et al., Radiation Oncology 2014, 9:200).

As described herein, prostate volume as a biomarker, in combination with FLNA and age, is a predictor of BPH progression. In some embodiments, a prostate size greater than 30 mL, 35 mL, 40 mL, or greater is considered enlarged. Since PSA increases with prostate size, prostate volume is important to assist with distinguishing BPH from PSA, along with the other biomarkers described herein, e.g., age and FLNA level.

In accordance with various embodiments, algorithms may be employed to predict whether or not a biological sample is likely to be diseased (e.g., having prostate cancer or BPH) or to differentiate between BPH and prostate cancer. The skilled artisan will appreciate that an algorithm can be any computation, formula, statistical survey, nomogram, look-up table, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output,” e.g., a diagnosis of prostate cancer. Any suitable algorithm—whether computer-based or manual-based (e.g., look-up table)—is contemplated herein.

In certain embodiments, an algorithm of the invention used to predict whether a biological sample has prostate cancer or BPH or to differentiate between BPH and prostate cancer by producing a score on the basis of the detected level of FLNA in the sample in combination with the variables of age and/or prostate volume. In some embodiments, if the score is above a certain threshold score, then the biological sample has prostate cancer.

In certain embodiments, an algorithm of the invention used to predict whether a biological sample has prostate cancer or BPH by producing a score on the basis of the detected level of FLNA in the sample in combination with the variable of age and/or prostate volume, wherein if the score is below a certain threshold score, then the biological sample has BPH.

### D. Diagnostic and Prognostic Uses of the Invention

The invention provides methods for diagnosing an abnormal prostate state, e.g., BPH or an oncological disease state, e.g., prostate cancer, in a subject. In one embodiment, the invention provides methods for distinguishing between prostate cancer versus benign prostatic hyperplasia (BPH) in a subject, e.g., using a biomarker panel as described herein, e.g., FLNA, age and prostate volume. In another embodiment, the invention provides methods for avoiding an unnecessary prostate biopsy in a subject. In another embodiment, the invention provides methods for monitoring a subject suspected of having BPH or prostate cancer. In another embodiment, the invention provides methods for treating a subject for BPH, e.g., following a diagnosis of BPH in the subject using the biomarkers of the invention.

As used herein the disorder, disease, or abnormal state is an abnormal prostate state, including LUTS, BPH and cancer, particularly prostate cancer. The prostate cancer may be a prostatic intraepithelial neoplasia, adenocarcinoma, small cell carcinoma, or squamous cell carcinoma.

The invention provides, in one embodiment, methods for diagnosing BPH, monitoring BPH, and differentiating between BPH and prostate cancer. The methods of the present invention can be practiced in conjunction with any other method used by the skilled practitioner to provide a prognosis of the occurrence or recurrence of BPH. The diagnostic and prognostic methods provided herein can be used to determine if additional and/or more invasive tests or monitoring should be performed on a subject, and can be used to avoid unnecessary invasive tests, e.g., biopsy. It is understood that a disease as complex as an abnormal prostate state is rarely diagnosed using a single test. Therefore, it is understood that the diagnostic, prognostic, and monitoring methods provided herein are typically used in conjunction with other methods known in the art. For example, the methods of the invention may be performed in conjunction with PSA screening, and/or physical exam, e.g., DRE. Cytological methods would include immunohistochemical or immunofluorescence detection (and quantitation if appropriate) of any other molecular marker either by itself, in conjunction with other markers. Other methods would include detection of other markers by in situ PCR, or by extracting tissue and quantitating other markers by real time PCR. PCR is defined as polymerase chain reaction. Exemplary screening paradigms using the biomarker panels of the invention are shown in FIG. 5B.

Methods for assessing or monitoring BPH during watchful waiting, following one or more negative biopsy, or during the efficacy of a treatment regimen are also provided. In some embodiments, the amount of marker in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of marker levels. Pairwise comparisons can be made between consecutive or non-consecutive subject samples. Trends of marker levels and rates of change of marker levels can be analyzed for any two or more consecutive or non-consecutive subject samples.

In particular embodiments, the invention provides methods for differentiating between BPH and prostate cancer in a subject by detecting the protein level of Filamin A (FLNA) in a biological sample from the subject; measuring the prostate volume of the subject; and analyzing the age of the subject, the prostate volume of the subject, and the protein level of FLNA in the biological sample with a corresponding predetermined threshold value for FLNA level, age and prostate volume. In preferred embodiments, the detection reagent is an anti-FLNA antibody, or an antigen-binding portion thereof. In other preferred embodiments, the detection method is IPMRM.

Optionally, additional prostate cancer-related markers can be detected such as PSA, keratin 19 (KRT19), and/or filamin B (FLNB) in the methods of the invention. Additional markers, including filamin B and keratin 19, and uses thereof in the diagnosis and prognosis of prostate cancer, are described in PCT Publication Nos. WO 2014/004931, filed on Jun. 27, 2013, and WO 2016/094425, filed on Dec. 8, 2015, the contents of which are expressly incorporated herein by reference.

The invention provides method of avoiding unnecessary biopsy in a subject by detecting the protein level of Filamin A (FLNA) in a biological sample from the subject; measuring the prostate volume of the subject; and analyzing the age of the subject, the prostate volume of the subject, and the protein level of FLNA in the biological sample with a corresponding predetermined threshold value for FLNA level, age and prostate volume. In preferred embodiments, the detection reagent is an anti-FLNA antibody, or an antigen-binding portion thereof. In other preferred embodiments, the detection method is IPMRM.

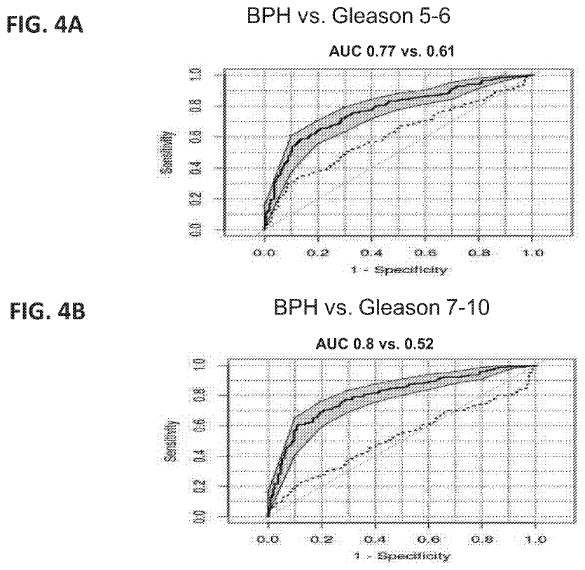

In one embodiment, the method using a biomarker panel comprising FLNA, age and prostate volume provides a predictive score, or AUC, of about 0.75 versus 0.55 for PSA in subjects who have had at least one biopsy (see FIG. 1B). In another embodiment, the method using a biomarker panel comprising FLNA, age and prostate volume provides a predictive score, or AUC, of about 0.87 versus 0.57 for PSA in subjects who have had multiple biopsies (see FIG. 2B). In another embodiment, the method using a biomarker panel comprising FLNA, age and prostate volume provides a predictive score, or AUC, of about 0.76 versus 0.56 for PSA in subjects with an intermediate Gleason score (5-7) (see FIG. 3A). In another embodiment, the method using a biomarker panel comprising FLNA, age and prostate volume provides a predictive score, or AUC, of about 0.74 versus 0.47 for PSA in subjects with a high Gleason score (≥8) (see FIG. 3B). In another embodiment, the method using a biomarker panel comprising FLNA, age and prostate volume provides a predictive score, or AUC, of about 0.77 versus 0.61 for PSA in subjects with an intermediate Gleason score (5-6) (see FIG. 4A). In another embodiment, the method using a biomarker panel comprising FLNA, age and prostate volume provides a predictive score, or AUC, of about 0.8 versus 0.52 for PSA in subjects with a high Gleason score (7-10) (see FIG. 4B).

In certain embodiments the diagnostic and monitoring methods provided herein further comprise comparing the detected level of the one or more prostate markers in the biological samples with one or more control samples wherein the control sample is one or more of a sample from the same subject at an earlier time point than the biological sample, or a sample from a subject without an abnormal prostate state. Comparison of the marker levels in the biological samples with control samples from subjects with various normal and abnormal prostate states facilitates the differentiation between benign prostate hyperplasia and prostate cancer.

In certain embodiments the diagnostic and monitoring methods provided herein further comprising detecting the size of prostate, e.g., by DRE.

In certain embodiments the diagnostic and monitoring methods provided herein further comprising obtaining a subject sample.

In certain embodiments the diagnostic and monitoring methods provided herein further comprising selecting a subject for having or being suspected of having prostate cancer.

In certain embodiments the diagnostic and monitoring methods provided herein further comprising treating the subject for BPH or symptoms thereof, e.g., LUTS, with a regimen including one or more treatments selected from the group consisting of a selective α₁-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

In certain embodiments the diagnostic and monitoring methods provided herein further comprising selecting the one or more specific treatment regimens for the subject based on the results of the diagnostic and monitoring methods provided herein. In certain embodiments, the treatment method is maintained based on the results from the diagnostic or prognostic methods. In certain embodiments, the treatment method is changed based on the results from the diagnostic or prognostic methods. In one embodiment, the biological sample is a serum sample

In certain embodiments of the diagnostic and monitoring methods provided herein, the method of detecting a level comprises isolating a component of the biological sample. In one embodiment, the biological sample is selected from the group consisting of blood, serum, plasma, and urine.

In certain embodiments of the diagnostic and monitoring methods provided herein, the method of detecting a level comprises labeling a component of the biological sample.

In certain embodiments of the diagnostic and monitoring methods provided herein, the method of detecting a level comprises amplifying a component of a biological sample.

In certain embodiments of the diagnostic and monitoring methods provided herein, the age of the subject is 50 years or older.

In certain embodiments of the diagnostic and monitoring methods provided herein, the subject is experiencing lower urinary tract symptoms (LUTS).

In certain embodiments of the diagnostic and monitoring methods provided herein, the subject has an enlarged prostate gland as determined by digital rectal examination (DRE).

In certain embodiments of the diagnostic and monitoring methods provided herein, the subject does not have an enlarged prostate gland as determined by digital rectal examination (DRE).

In certain embodiments of the diagnostic and monitoring methods provided herein, the subject has an elevated prostate specific antigen (PSA) level, e.g., between 4-10 ng/mL.

In certain embodiments of the diagnostic and monitoring methods provided herein, the subject has had one or more prostate biopsies.

In certain embodiments of the diagnostic and monitoring methods provided herein, BPH is differentiated from prostate cancer in a subject having an intermediate Gleason score of from 5 to 7.

In certain embodiments of the diagnostic and monitoring methods provided herein, BPH is differentiated from prostate cancer in a subject having a high Gleason score of greater than 8.

In certain embodiments of the diagnostic and monitoring methods provided herein, BPH is differentiated from prostate cancer in a subject having an intermediate Gleason score of from 5 to 6.

In certain embodiments of the diagnostic and monitoring methods provided herein, BPH is differentiated from prostate cancer in a subject having a high Gleason score of 7-10.

In certain embodiments of the diagnostic and monitoring methods provided herein, the method of detecting a level comprises forming a complex with a probe and a component of a biological sample. In certain embodiments, forming a complex with a probe comprises forming a complex with at least one non-naturally occurring reagent. In certain embodiments of the diagnostic and monitoring methods provided herein, the method of detecting a level comprises processing the biological sample. In certain embodiments of the diagnostic and monitoring methods provided herein, the method of detecting a level of at least two markers comprises a panel of markers. In certain embodiments of the diagnostic and monitoring methods provided herein, the method of detecting a level comprises attaching the marker to be detected to a solid surface.

### 1. Diagnostic Assays

An exemplary method for detecting the presence or absence or change of expression level of a marker protein or nucleic acid in a biological sample involves obtaining a biological sample (e.g. an oncological disorder-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. In a preferred embodiment, the binding agent is an FLNA binding protein, e.g., antibody, or antigen binding fragment thereof, as described herein.

Methods provided herein for detecting the presence, absence, change of expression level of a marker protein or nucleic acid in a biological sample include obtaining a biological sample from a subject that may or may not contain the marker protein to be detected, contacting the sample with a marker-specific binding agent (i.e., a FLNA binding protein, e.g., antibody, or antigen binding fragment thereof, as described herein) that is capable of forming a complex with the marker protein, and contacting the sample with a detection reagent for detection of the marker-marker-specific binding agent complex, if formed. It is understood that the methods provided herein for detecting an expression level of a marker in a biological sample includes the steps to perform the assay. In certain embodiments of the detection methods, the level of the marker protein or nucleic acid in the sample is none or below the threshold for detection.

The methods include formation of either a transient or stable complex between the marker and the marker-specific binding agent (e.g., a FLNA antibody, or antigen binding fragment thereof as described herein). The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

In certain embodiments, all markers are detected using the same method. In certain embodiments, all markers are detected using the same biological sample (e.g., same body fluid or tissue). In certain embodiments, different markers are detected using various methods. In certain embodiments, markers are detected in different biological samples.

### E. Detection and/or Measurement of Biomarkers

The present invention contemplates any suitable means, techniques, and/or procedures for detecting and/or measuring the biomarkers of the invention. The skilled artisan will appreciate that the methodologies employed to measure the biomarkers of the invention will depend at least on the type of biomarker being detected or measured (e.g., mRNA biomarker or polypeptide biomarker) and the source of the biological sample (e.g., whole blood versus plasma). In a preferred embodiment, FLNA level in a sample is measured by detecting FLNA polypeptide using an IPMRM assay.

### 1. Detection of Polypeptide Biomarkers

The present invention contemplates any suitable method for detecting polypeptide biomarkers of the invention. In certain embodiments, the detection method is an immunodetection or immunoassay method involving a binding protein, e.g., an antibody, that specifically binds to one or more of the biomarkers of the invention, e.g., FLNA. The steps of various useful immunodetection methods have been described in the scientific literature, such as, e.g., Nakamura et al. (1987), which is incorporated herein by reference.

In general, the immunobinding methods include obtaining a sample suspected of containing a biomarker protein, peptide or antibody, and contacting the sample with a binding protein, e.g., an antibody in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a prostate specific protein, peptide or a corresponding antibody, and contact the sample with an binding protein, e.g., an antibody, or encoded protein or peptide, as the case may be, and then detect or quantify the amount of immune complexes formed under the specific conditions. In terms of biomarker detection, the biological sample analyzed may be any sample that is suspected of containing a biomarker, such as, FLNA. The biological sample may be, for example, a serum sample, a prostate or lymph node tissue section or specimen, a homogenized tissue extract, an isolated cell, a cell membrane preparation, separated or purified forms of any of the above protein-containing compositions, or any biological fluid including blood, plasma, or lymphatic fluid.

Contacting the chosen biological sample with the protein (e.g., FLNA or antigen thereof to bind with an anti-FLNA antibody in the blood), peptide (e.g., FLNA fragment that binds with an anti-FLNA antibody in the blood), or binding protein, e.g., an antibody (e.g., as a detection reagent that binds FLNA in a biological sample) under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein (e.g., FLNA), peptide (e.g., FLNA peptide) or corresponding antibody (anti-FLNA antibody as detection reagent) employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as benign prostatic hyperplasia or prostate cancer. Here, a biological or clinical sample suspected of containing either the encoded FLNA protein or peptide or corresponding antibody is used. However, these embodiments also have applications to non-clinical samples, such as in the tittering of antigen or antibody samples, in the selection of hybridomas, and the like.

In some embodiments, the use of ELISAs as a type of immunodetection assay is contemplated. In some embodiments, the biomarker proteins or peptides of the invention will find utility as immunogens in ELISA assays in diagnosis and prognostic monitoring of benign prostatic hyperplasia (BPH). Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the biomarkers of the invention are immobilized onto a selected surface exhibiting protein affinity, such as a well in a polystyrene microtiter plate. Then, a test composition suspected of containing the prostate cancer marker antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the prostate cancer marker antigen are immobilized onto the well surface and then contacted with the anti-biomarker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human prostate, cancer and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

The protein biomarkers of the invention (e.g., FLNA) can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of the biomarkers of the invention can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The biomarkers of the invention can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

The biomarkers of the present invention (e.g., FLNA) can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (13C) or nitrogen (15N) can be incorporated into one sample while the other one can be labeled with corresponding light isotopes (e.g. 12C and 14N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed 180 labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

In one embodiment, any one or more of the biomarkers of the invention (e.g., FLNA) can be identified and quantified from a complex biological sample using mass spectroscopy in accordance with the following exemplary method, which is not intended to limit the invention or the use of other mass spectrometry-based methods.

Proteins are detected using a number of assays in which a complex between the marker protein to be detected and the marker specific binding agent would not occur naturally, for example, because one of the components is not a naturally occurring compound or the marker for detection and the marker specific binding agent are not from the same organism (e.g., human marker proteins detected using marker-specific binding antibodies from mouse, rat, or goat). In a preferred embodiment of the invention, the marker protein for detection is a human marker protein. In certain detection assays, the human markers for detection are bound by marker-specific, non-human antibodies, thus, the complex would not be formed in nature. The complex of the marker protein can be detected directly, e.g., by use of a labeled marker-specific antibody that binds directly to the marker, or by binding a further component to the marker-specific antibody complex. In certain embodiments, the further component is a second marker-specific antibody capable of binding the marker at the same time as the first marker-specific antibody. In certain embodiments, the further component is a secondary antibody that binds to a marker-specific antibody, wherein the secondary antibody preferably linked to a detectable label (e.g., fluorescent label, enzymatic label, biotin). When the secondary antibody is linked to an enzymatic detectable label (e.g., a peroxidase, a phosphatase, a beta-galactosidase), the secondary antibody is detected by contacting the enzymatic detectable label with an appropriate substrate to produce a colorimetric, fluorescent, or other detectable, preferably quantitatively detectable, product. Antibodies for use in the methods of the invention can be polyclonal, however, in a preferred embodiment monoclonal antibodies are used. An intact antibody, or a fragment or derivative thereof (e.g., Fab or F(ab′)₂) can be used in the methods of the invention. Such strategies of marker protein detection are used, for example, in ELISA, RIA, immunoprecipitation and western blot, Immunoprecipitation-Multiple Reaction Monitoring (IPMRM) or LC-MS/MS, and immunofluorescence assay methods.

In certain embodiments, the marker-specific binding agent complex is attached to a solid support for detection of the marker. The complex can be formed on the substrate or formed prior to capture on the substrate. For example, in an ELISA, RIA, immunoprecipitation assay, western blot, immunofluorescence assay, in gel enzymatic assay the marker for detection is attached to a solid support, either directly or indirectly. In an ELISA, RIA, or immunofluorescence assay, the marker is typically attached indirectly to a solid support through an antibody or binding protein.

In a western blot or immunofluorescence assay, the marker is typically attached directly to the solid support. For in-gel enzyme assays, the marker is resolved in a gel, typically an acrylamide gel, in which a substrate for the enzyme is integrated.

In another aspect, this application provides methods for detecting the presence, absence, change of expression level of FLNA using an immunoaffinity enrichment approach coupled with MRM (i.e., IPMRM). IPMRM assays for detecting FLNA are described in U.S. patent application Ser. No. 15/801,093, filed on Nov. 1, 2017, the contents of which are hereby incorporated herein by reference.

IPMRM combines immunoprecipitation (IP) with mass spectrometry and allows for the rapid quantitation of proteins with enhanced sensitivity and specificity. For biomarkers, this technique has been shown to achieve low mg/mL quantitation by selective enrichment of target proteins in complex matrices (see Nicol G R, et al. (2008) Molecular & Cellular Proteomics 7 (10):1974-1982; Kulasingam V, et al. (2008) Journal of Proteome Research 7 (2):640-647; Berna M, Ackermann B (2009) Anal Chem 81 (10):3950-3956, the contents of which are incorporated herein by reference). For example, ELISA alone may not detect all forms of FLNA in a sample. However, IPMRM allows detection of different peptides along the length of the entire protein and thus has increased specificity. In one embodiment, IPMRM is used for the detection of FLNA in a serum sample.

In one embodiment, IPMRM is used for the detection of FLNA in a plasma sample.

In one embodiment, IPMRM involves enrichment of one or more markers, e.g., FLNA, using one or more capture antibodies (e.g., one or more of the binding proteins as described below), followed by digestion and analysis of surrogate peptides by stable isotope dilution MRM. For example, one or more of the 2C12, 3F4, and/or 6E3 antibodies, as described herein, may be used as the capture antibodies in the methods of the invention. In one embodiment, the 2C12 and 3F4 antibodies are used as the capture antibodies in the assay. In another embodiment, surrogate peptides can be tryptic peptides between, for example, 8 and 22 amino acids. In one embodiment, surrogate peptides used in FLNA IPMRM can comprise one or more of peptides P2 (AGVAPLQVK) (SEQ ID NO:35) and P4 (YNEQHVPGSPFTAR) (SEQ ID NO:36). In one embodiment, peptide P2 is used in the IPMRM.

In particular, for IPMRM, capture antibodies (e.g., one or more of the 2C12, 3F4, and/or 6E3 antibodies described herein), are immobilized onto a support using methods known in the art, e.g., onto an agarose support using, for example, the ThermoFisher Scientific Pierce Direct IP Kit (ThermoFisher Scientific), and coupled to coupling resin. Immunoprecipitation can then be performed using methods known in the art. For example, the Pierce Direct IP Kit can be used. In one embodiment, the resin-coupled antibodies can be washed and human serum added along with prepared lysis buffer solution and EDTA, and incubated. The resin can then be washed again with IP lysis/wash buffer and conditioning buffer. The captured proteins can then be eluted and incubated. The IP eluates from the surrogate matrix can be used to prepare peptide (e.g., P2 and/or P4) calibration curves by spiking with a synthetic peptide stock solution. Samples can then by subjected to trypsin digestion using methods known in the art (e.g., using the Flash Digest Kit (Perfinity Biosciences, West Lafayette, IN).

MRM analysis can be performed on a mass spectrometer, e.g., a 6500 QTRAP mass spectrometer (Sciex) equipped with an electrospray source, a 1290 Infinity UPLC™ system (Agilent Technologies, Santa Clara, CA) and a XBridge® Peptide BEH300 C18 (3.5 μm, 2.1 mm×150 mm) column (Waters, Milford, MA). Liquid chromatography can then be carried out. For example, liquid chromatography can be carried out at a flow rate of 400 μL/min, with a sample injection volume of 30 μL at a temperature of 60° C. In one embodiment, mobile phase A can consist of 0.1% formic acid (Sigma Aldrich) in water (ThermoFisher Scientific) and mobile phase B can consist of 0.1% formic acid in acetonitrile (ThermoFisher Scientific). The gradient with respect to % B can be as follows: 0-1.5 min, 5%; 1.5-2 min, 5-15%; 2-5 min, 15%; 5-7.1 min, 15-20%; 7.1-8.1 min, 20-80%; 8.1-9.0 min, 80%; and 9.0-9.1 min, 80-5%, 9.1-16 min, 5%.

In one embodiment, the instrument parameters for the mass spectrometer, e.g., a 6500 QTRAP mass spectrometer, can be as follows: Ion spray voltage of 5500 V, curtain gas of 20 psi, collision gas set to “medium”, interface heater temperature of 400° C., nebulizer gas (GS1) of 80 psi and ion source gas (GS2) of 80 psi and unit resolution for both Q1 and Q3 quadrupoles.

Potential surrogate peptides for FLNA quantitation can be chosen by methods known in the art, e.g., using Skyline® software and LC-MS/MS analysis (LTQ Orbitrap Velos™ coupled to Eksigent nano-LC™) of recombinant FLNA protein (GenScript®) tryptic digest. Surrogate peptides can be chosen based on surrogate peptide selection rules (Halquist, et al., Biomed Chromatography 25 (1-2):47-58) and signal intensities of the peptides in spiked and unspiked serum digests. The uniqueness of the surrogate peptides to the target protein can confirmed by running BLAST searches. In one embodiment, heavy labeled versions of surrogate peptide 2 (P2) and peptide 4 (P4), AGVAPLQV[K (13C6; 15N2)] (SEQ ID NO: 35) and YNEQHVPGSPFTA[R (13C6; 15N4)] (SEQ ID NO: 36), which were selected using the methods described above, can be used as internal standards.

MRM transitions can be optimized using synthetic surrogate peptides (GenScript®) and their internal standards (ThermoFisher Scientific) and the following m/z transitions can be monitored: 441.7 (M+2H)²⁺→584.5 (y₅¹⁺) for P2; 535 (M+3H)³⁺→832.4 (y₈¹⁺) for P4, 445.5 (M+2H)²⁺→592.1 (y₅¹⁺) for P2 internal standard (P2_IS), and 538.4 (M+3H)³⁺→842.5 (y₈¹⁺) for P4 internal standard P4 IS.

Analysis and quantitation of IPMRM data can be performed using methods known in the art, for example, the Analyst® software (version 1.6.2, AB Sciex, Framingham, MA). In one embodiment, peak integrations can be reviewed manually. The calibration curve for FLNA P2 and P4 peptides can be constructed by plotting the peak area ratios (analyte/internal standard) versus concentration of the standard with 1/x² linear least square regression.

In yet another aspect, this application provides a method for detecting the presence of FLNA in vivo (e.g., in vivo imaging in a subject). The subject method can be used to diagnose a disorder, e.g., an abnormal prostate stated, including BPH. In exemplary embodiments, the method includes: (i) administering the anti-FLNA antibody or fragment thereof as described herein to a subject or a control subject under conditions that allow binding of the antibody or fragment to FLNA; and (ii) detecting formation of a complex between the antibody or fragment and FLNA, wherein a statistically significant change in the formation of the complex in the subject relative to the control subject is indicative of the presence of FLNA.

### 2. Detection of Nucleic Acid Biomarkers

In certain embodiments, the invention involves the detection of nucleic acid biomarkers, e.g., mRNA biomarkers of FLNA alone or FLNA in combination with at least one other prostate cancer related marker selected from the group consisting of filamin B, LY9, keratin 4, keratin 7, keratin 8, keratin 15, keratin 18, keratin 19, tubulin-beta 3, PSA, PSM, PSCA, TMPRSS2, PDEF, HPG-1, PCA3, and PCGEM1.

In various embodiments, the diagnostic/prognostic methods of the present invention generally involve the determination of expression levels of a set of genes in a prostate tissue sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding biomarkers of the invention, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86:5673-5677), in situ hybridization, Taqman-based assays (Holland et al., Proc. Natl. Acad. Sci., 1991, 88:7276-7280), differential display (see, for example, Liang et al., Nucl. Acid. Res., 1993, 21:3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of biomarkers of interest may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270:467-470; M. Schena et al., Proc. Natl. Acad. Sci. USA 1996, 93:10614-10619; J. J. Chen et al., Genomics, 1998, 51:313-324; U.S. Pat. Nos. 5,143,854; 5,445,934; 5,807,522; 5,837,832; 6,040,138; 6,045,996; 6,284,460; and 6,607,885).

In one particular embodiment, the invention comprises a method for identification of prostate cancer cells in a biological sample by amplifying and detecting nucleic acids corresponding to the novel prostate cancer biomarkers, and or panels of biomarkers that include FLNA alone or FLNA in combination with one or more markers selected from the group consisting of filamin B, LY9, keratin 4, keratin 7, keratin 8, keratin 15, keratin 18, keratin 19, tubulin-beta 3, PSA, PSM, PSCA, TMPRSS2, PDEF, HPG-1, PCA3, and PCGEM1. The biological sample may be any tissue or fluid in which prostate cancer cells might be present. Various embodiments include radical prostatectomy specimens, pathological specimens, bone marrow aspirate, bone marrow biopsy, lymph node aspirate, lymph node biopsy, spleen tissue, fine needle aspirate, skin biopsy or organ tissue biopsy. Other embodiments include samples where the body fluid is peripheral blood, serum, plasma, lymph fluid, ascites, serous fluid, pleural effusion, sputum, cerebrospinal fluid, lacrimal fluid, stool, prostatic fluid or urine.

Nucleic acid used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to any of the prostate cancer biomarker nucleotide sequences identified herein are contacted with the isolated nucleic acid under conditions that permit selective hybridization. Once hybridized, the nucleic acid: primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994). Following detection, one may compare the results seen in a given patient with a statistically significant reference group of normal patients and prostate, cancer patients. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[a-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA: RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32 . . . ) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention. In one embodiment, the oligonucleotide probes or primers of the present invention specifically hybridize with a FLNA RNA (or its complementary sequence) or a FLNA mRNA. More preferably, the FLNA primers and probes will be chosen to detect a FLNA RNA which is associated with prostate cancer.

In other embodiments, the detection means can utilize a hybridization technique, e.g., where a specific primer or probe is selected to anneal to a target biomarker of interest, e.g., FLNA, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of a FLNA or polynucleotide of another biomarker of the invention. Probes and primers of the present invention are those that hybridize under stringent hybridization conditions and those that hybridize to biomarker homologs of the invention under at least moderately stringent conditions. In certain embodiments probes and primers of the present invention have complete sequence identity to the biomarkers of the invention (FLNA, gene sequences (e.g., cDNA or mRNA). It should be understood that other probes and primers could be easily designed and used in the present invention based on the biomarkers of the invention disclosed herein by using methods of computer alignment and sequence analysis known in the art (cf. Molecular Cloning: A Laboratory Manual, Third Edition, edited by Cold Spring Harbor Laboratory, 2000).

### F. Anti FLNA Binding Proteins

The present invention features methods for detection of levels of FLNA which utilize binding proteins comprising an antigen binding domain, said binding protein capable of binding FLNA. Any FLNA binding proteins, e.g., human or murine anti-FLNA antibodies, that specifically bind to FLNA can be used in the methods of the invention to detect levels of FLNA in a sample. Exemplary FLNA binding proteins that can be used in the methods of the invention include the antibodies described in U.S. patent application Ser. No. 15/801,093, filed on Nov. 1, 2017, the contents of which are hereby incorporated herein by reference. A listing of amino acid sequences of VH and VL regions of these exemplary anti-FLNA monoclonal antibodies, i.e., murine FLNA antibodies 2C12, 3F4, and 6E3, are shown below in Table 1. The CDRs, as determined by the IMGT numbering system (Lefranc, M.-P. et al., Nucleic Acids Research, 27, 209-212 (1999)), are underlined, as shown below in Table 1.

TABLE 1
Amino Acid Sequences of VH and VL regions

| SEQ | | |
|---|---|---|
| ID No. | Clone-Protein | Sequence |
| 1 | 2C12 VH | QVQLKQSGPGLVQPSQSLSITCTV |
| | | SGFSLTNYGVHWVRQSPGKGLE |
| | | WLGVIWRGGSTDYNAAFMSRLSI |
| | | TKDNSKSQVFFKMNSLQADDTAI |
| | | YFCALRGNYVHYYLMDYWGQG |
| | | TSVTVSS |
| 7 | 2C12 VH CDR1 | GFSLTNYG |
| 8 | 2C12 VH CDR2 | IWRGGST |
| 9 | 2C12 VH CDR3 | ALRGNYVHYYLMDY |
| 2 | 2C12 VL | DIQVTQTPSSLSASLGDRVTISCRA |
| | | SQDISNYLNWYQQKPDGTVKLLI |
| | | YYTSRLHSGVPSRFSGSGSGTDYS |
| | | LTISNLDQEDIATYFCQQGNTLPP |
| | | TFGGGTNLEIK |
| 10 | 2C12 VL CDR1 | QDISNY |
| 11 | 2C12 VL CDR2 | YTS |
| 12 | 2C12 VL CDR3 | QQGNTLPPT |
| 3 | 3F4 VH | EVQLQESGPGLAKPSQTLSLTCSV |
| | | TGYSITSNYWNWIRKFPGNKLEY |
| | | MGYISFSGSTYYNPSLKSRISITRD |
| | | TSKNQYYLQLNSVTTEDTATYYC |
| | | ARWNYYAMDYWGQGTSVTVSS |
| 13 | 3F4 VH CDR1 | GYSITSNY |
| 14 | 3F4 VH CDR2 | ISFSGST |
| 15 | 3F4 VH CDR3 | ARWNYYAMDY |
| 4 | 3F4 VL | DFLLTQSPAILSVSPGERVSFSCRA |
| | | SQSIGTNIHWYQQRTNGSPRLLIK |
| | | FASESISGIPSRFSGSGSGTDFTLTI |
| | | NSVESEDIADYYCQQSNSWPYTF |
| | | GGGTKLEIK |
| 16 | 3F4 VL CDR1 | QSIGTN |
| 17 | 3F4 VL CDR2 | FAS |
| 18 | 3F4 VL CDR3 | QQSNSWPYT |
| 5 | 6E3 VH | QVQLQQSGAELMKPGASVKLSC |
| | | KATGYTFTGYWIEWVKQRPGHG |
| | | LEWIGEILPGNGSTNCNEKFKGKA |
| | | TFTATTSSNTAYMQLSSLTTEDSA |
| | | IYYCTTVSYWGQGTTLTVSS |
| 19 | 6E3 VH CDR1 | GYTFTGYW |
| 20 | 6E3 VH CDR2 | ILPGNGST |
| 21 | 6E3 VH CDR3 | TTVSY |
| 6 | 6E3 VL | DVVMTQTPLSLPVSLGDQASISCR |
| | | SSQSLVHSNGNTYLHWYLQKPGQ |
| | | SPNLLIYKVSNRFSGVPDRFTGSG |
| | | SGTDFTLKISRVEAEDLGVYFCSQ |
| | | STHVPFTFGSGTKLEIK |
| 22 | 6E3 VL CDR1 | QSLVHSNGNTY |
| 23 | 6E3 VL CDR2 | KVS |
| 24 | 6E3 VL CDR3 | SQSTHVPFT |

The present invention features in other aspects, methods for using a binding protein comprising an antigen binding domain, said binding protein capable of binding filamin A (FLNA), said antigen binding domain comprising a heavy chain variable region comprising a CDR3 domain comprising the amino acid sequence of SEQ ID NO: 9, a CDR2 domain comprising the amino acid sequence of SEQ ID NO: 8, and a CDR1 domain comprising the amino acid sequence of SEQ ID NO: 7, and a light chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10.

The present invention also features in other aspects, methods for using a binding protein comprising an antigen binding domain, said binding protein capable of binding filamin A (FLNA), said antigen binding domain comprising a heavy chain variable region comprising a CDR3 domain comprising the amino acid sequence of SEQ ID NO: 15, a CDR2 domain comprising the amino acid sequence of SEQ ID NO: 14, and a CDR1 domain comprising the amino acid sequence of SEQ ID NO: 13, and a light chain variable region comprising a CDR3 domain comprising the amino acid sequence of SEQ ID NO: 18, a CDR2 domain comprising the amino acid sequence of SEQ ID NO: 17, and a CDR1 domain comprising the amino acid sequence of SEQ ID NO: 16.

The present invention also features in other aspects, methods for using a binding protein comprising an antigen binding domain, said binding protein capable of binding filamin A (FLNA), said antigen binding domain comprising a heavy chain variable region comprising a CDR3 domain comprising the amino acid sequence of SEQ ID NO: 21, a CDR2 domain comprising the amino acid sequence of SEQ ID NO: 20, and a CDR1 domain comprising the amino acid sequence of SEQ ID NO: 19, and a light chain variable region comprising a CDR3 domain comprising the amino acid sequence of SEQ ID NO: 24, a CDR2 domain comprising the amino acid sequence of SEQ ID NO: 23, and a CDR1 domain comprising the amino acid sequence of SEQ ID NO: 22.

In one embodiment of the above aspects, the antigen binding domain comprises a heavy chain variable region selected from the group consisting of: the amino acid sequence set forth in SEQ ID NO: 1, the amino acid sequence set forth in SEQ ID NO: 3 or the amino acid sequence set forth in SEQ ID NO: 5.

In another embodiment of the above aspects, the antigen binding domain comprises a light chain variable region selected from the group consisting of: the amino acid sequence set forth in SEQ ID NO: 2, the amino acid sequence set forth in SEQ ID NO: 4 or the amino acid set forth in SEQ ID NO: 6.

In one embodiment of the above aspects, the antigen binding domain comprises a heavy chain variable region comprising the amino acid sequence set forth in SEQ ID NO: 1 and a light chain variable region comprising the amino acid sequence set forth in SEQ ID NO: 2.

In another embodiment of the above aspects, the antigen binding domain comprises a heavy chain variable region comprising the amino acid sequence set forth in SEQ ID NO: 3 and a light chain variable region comprising the amino acid sequence set forth in SEQ ID NO: 4.

In another embodiment of the above aspects, the antigen binding domain comprises a heavy chain variable region comprising the amino acid sequence set forth in SEQ ID NO: 5 and a light chain variable region comprising the amino acid sequence set forth in SEQ ID NO: 6.

In certain embodiments, the term “2C12” refers to a hybridoma that produces an antibody comprising (i) one variable heavy chain having an amino acid sequence comprising SEQ ID NO: 1; and (ii) one variable light chain having an amino acid sequence comprising SEQ ID NO: 2. In certain embodiments, the 2C12 heavy chain variable region comprises a CDR3 domain comprising the amino acid sequence of SEQ ID NO: 9, a CDR2 domain comprising the amino acid sequence of SEQ ID NO: 8, and a CDR1 domain comprising the amino acid sequence of SEQ ID NO: 7, and the light chain variable region comprises a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10. In certain embodiments, antibody 2C12 can have an on rate constant (KON) to FLNA of at least about 1×10⁴ M⁻¹ s⁻¹ to about 6×10⁶ M⁻¹ s⁻¹ or about 5×10⁴ M⁻¹ s⁻¹ to about 9×10⁵ M⁻¹ s⁻¹ as measured by surface plasmon resonance. In other embodiments, the binding protein according to the present invention can have an on rate constant (KON) to FLNA of least about 7.9×10⁴ M⁻¹ s⁻¹ as measured by surface plasmon resonance. In other embodiments, the binding protein according to the present invention can have a dissociation constant (K_{D}) to FLNA of 4.82×10⁻⁹ s⁻¹ or less. In certain preferred embodiments, the binding protein according to the present invention has a dissociation constant (K_{D}) to FLNA of about 1.0×10⁻⁷ s⁻¹ or less, or about 1×10⁻⁸ M or less. According to preferred embodiments of the invention, the isotype of the antibody construct produced by the 2C12 hybridoma clone is IgG1/κ.

In other certain embodiments, the term “3F4” refers to a hybridomas that produces an antibody comprising (i) one variable heavy chain having an amino acid sequence comprising SEQ ID NO: 3; and (ii) one variable light chain having an amino acid sequence comprising SEQ ID NO: 4. In certain embodiments, the 3F4 heavy chain variable region comprises a CDR3 domain comprising the amino acid sequence of SEQ ID NO: 15, a CDR2 domain comprising the amino acid sequence of SEQ ID NO: 14, and a CDR1 domain comprising the amino acid sequence of SEQ ID NO: 13, and the light chain variable region comprises a CDR3 domain comprising the amino acid sequence of SEQ ID NO: 18, a CDR2 domain comprising the amino acid sequence of SEQ ID NO: 17, and a CDR1 domain comprising the amino acid sequence of SEQ ID NO: 16. In certain embodiments, the antibody 3F4 can have an on rate constant (KON) to FLNA of at least about 1×10⁴ M⁻¹ s⁻¹ to about 6×10⁶ M⁻¹ s⁻¹ or about 5×10⁴ M⁻¹ s⁻¹ to about 9×10⁵ M⁻¹ s⁻¹ as measured by surface plasmon resonance. In other embodiments, the binding protein according to the present invention can have an on rate constant (KON) to FLNA of at least about 8.05×10⁵ M⁻¹ s⁻¹ as measured by surface plasmon resonance. In other embodiments, the binding protein according to the present invention can have a dissociation constant (K_{D}) to FLNA of 9.99×10⁻¹⁰ s⁻¹ or less. In certain preferred embodiments, the binding protein according to the present invention has a dissociation constant (K_{D}) to FLNA of about 1.0×10⁻⁷ s⁻¹ or less, or about 1×10⁻⁸ M or less. According to other preferred embodiments of the invention, the isotype of the antibody construct produced by the 3F4 hybridoma clone is IgG2B/κ.

In other certain embodiments, the term “6E3” refers to a hybridomas that produces an antibody comprising (i) one variable heavy chain having an amino acid sequence comprising SEQ ID NO: 5; and (ii) one variable light chain having an amino acid sequence comprising SEQ ID NO: 6. In certain embodiments, the 6E3 heavy chain variable region comprises a CDR3 domain comprising the amino acid sequence of SEQ ID NO: 21, a CDR2 domain comprising the amino acid sequence of SEQ ID NO: 20, and a CDR1 domain comprising the amino acid sequence of SEQ ID NO: 19, and the light chain variable region comprises a CDR3 domain comprising the amino acid sequence of SEQ ID NO: 24, a CDR2 domain comprising the amino acid sequence of SEQ ID NO: 23, and a CDR1 domain comprising the amino acid sequence of SEQ ID NO: 22. In certain embodiments, antibody 6E3 can have an on rate constant (KON) to FLNA of at least about 1×10⁴ M⁻¹ s⁻¹ to about 6×10⁶ M⁻¹ s⁻¹ or about 5×10⁴ M⁻¹ s⁻¹ to about 9×10⁵ M⁻¹ s⁻¹ as measured by surface plasmon resonance. In other embodiments, the binding protein according to the present invention can have an on rate constant (KON) to FLNA of at least about 1.95×10⁵ M⁻¹ s⁻¹ as measured by surface plasmon resonance. In other embodiments, the binding protein according to the present invention can have a dissociation constant (K_{D}) to FLNA of 4.09×10⁻⁹ s⁻¹ or less. In certain preferred embodiments, the binding protein according to the present invention has a dissociation constant (K_{D}) to FLNA of about 1.0×10⁻⁷ s⁻¹ or less, or about 1×10⁻⁸ M or less. According to other preferred embodiments of the invention, the isotype of the antibody construct produced by the 6E3 hybridoma clone is IgG1/κ.

In one embodiment, the antigen binding domain comprises a heavy chain comprising the amino acid sequence set forth in SEQ ID NO: 25, and a light chain comprising the amino acid sequence set forth in SEQ ID NO: 26.

In another embodiment, the antigen binding domain comprises a heavy chain comprising the amino acid sequence set forth in SEQ ID NO: 27, and a light chain comprising the amino acid sequence set forth in SEQ ID NO: 28.

In another embodiment, the antigen binding domain comprises a heavy chain comprising the amino acid sequence set forth in SEQ ID NO: 29, and a light chain comprising the amino acid sequence set forth in SEQ ID NO: 30.

In certain embodiments, the heavy chain consensus amino acid sequence produced by the 2C12 hybridoma comprises SEQ ID NO: 25, shown below. In SEQ ID NO: 25, the variable heavy domain is highlighted in bold.

SEQ ID NO: 25
MAVLGLLFCLVTFPSCVLSQVQLKQSGPGLVQPSQSLSITCTVSGFSLTN
YGVHWVRQSPGKGLEWLGVIWRGGSTDYNAAFMSRLSITKDNSKSQVFFK
MNSLQADDTAIYFCALRGNYVHYYLMDYWGQGTSVTVSSAKTTPPSVYPL
AP

In certain embodiments, the light chain consensus amino acid sequence produced by the 2C12 hybridoma comprises SEQ ID NO: 26, shown below. In SEQ ID NO: 26, the variable light domain is highlighted in bold.

SEQ ID NO: 26
MVSTAQFLGLLLLCFQGTRCDIQVTQTPSSLSASLGDRVTISCRASQDIS
NYLNWYQQKPDGTVKLLIYYTSRLHSGVPSRFSGSGSGTDYSLTISNLDQ
EDIATYFCQQGNTLPPTFGGGTNLEIKRADAAPTVSIFPPSSEQLTSGGA
SVVCFLNNFYPK

In certain embodiments, the heavy chain consensus amino acid sequence produced by the 3F4 hybridoma comprises SEQ ID NO: 27, shown below. In SEQ ID NO: 27, the variable heavy domain is highlighted in bold.

SEQ ID NO: 27
MMVLSLLYLLTALPGILSEVQLQESGPGLAKPSQTLSLTCSVTGYSITSN
YWNWIRKFPGNKLEYMGYISFSGSTYYNPSLKSRISITRDTSKNQYYLQL
NSVTTEDTATYYCARWNYYAMDYWGQGTSVTVSSAKTTPPSVFPLA

In certain embodiments, the light chain consensus amino acid sequence produced by the 3F4 hybridoma comprises SEQ ID NO: 28, shown below. In SEQ ID NO: 28, the variable light domain is highlighted in bold.

SEQ ID NO: 28
MVSTAQFLVFLLFWIPASRGDFLLTQSPAILSVSPGERVSFSCRASQSIG
TNIHWYQQRTNGSPRLLIKFASESISGIPSRFSGSGSGTDFTLTINSVES
EDIADYYCQQSNSWPYTFGGGTKLEIKRADAAPTVSIFPPSSEQLTSGGA
SVVCFLNNFYPR

In certain embodiments, the heavy chain consensus amino acid sequence produced by the 6E3 hybridoma comprises SEQ ID NO: 29, shown below. In SEQ ID NO: 29, the variable heavy domain is highlighted in bold.

SEQ ID NO: 29
MGWSWVMLFLLSVTAGVHSQVQLQQSGAELMKPGASVKLSCKATGYTFTG
YWIEWVKQRPGHGLEWIGEILPGNGSTNCNEKFKGKATFTATTSSNTAYM
QLSSLTTEDSAIYYCTTVSYWGQGTTLTVSSAKTTPPSVFPLA

In certain embodiments, the light chain consensus amino acid sequence produced by the 6E3 hybridoma comprises SEQ ID NO: 30, shown below. In SEQ ID NO: 30, the variable light domain is highlighted in bold.

SEQ ID NO: 30
MKLPVRLLVLMFWIPASTSDVVMTQTPLSLPVSLGDQASISCRSSQSLVHS
NGNTYLHWYLQKPGQSPNLLIYKVSNRFSGVPDRFTGSGSGTDFTLKISRV
EAEDLGVYFCSQSTHVPFTFGSGTKLEIKRADAAPTVSIFPPSSEQLTSGG
ASVVCFLNNFYPK

According to preferred embodiments of the present invention, the binding protein as described herein is an antibody.

The present invention features an antibody construct comprising a binding protein as described herein, wherein the antibody construct further comprises a linker polypeptide or an immunoglobulin constant domain.

The antibody construct used in the methods of the present invention may comprise a heavy chain immunoglobulin constant domain selected from the group consisting of a IgM constant domain, a IgG4 constant domain, a IgG1 constant domain, a IgE constant domain, a IgG2 constant domain, a IgG3 constant domain and a IgA constant domain.

In certain embodiments, the binding protein used in the methods of the present invention comprises an IgG1 constant domain. In other embodiments, the binding protein used in the methods of the present invention comprises an IgG2 constant region, preferably IgG2b.

Furthermore, the antibody used in the methods of the present invention can comprise a light chain constant region, either a kappa light chain constant region or a lambda light chain constant region. Preferably, the antibody comprises a kappa light chain constant region. According to preferred embodiments of the invention, the isotype of the antibody construct produced by the 2C12 hybridoma clone is IgG1/κ. According to other preferred embodiments of the invention, the isotype of the antibody construct produced by the 3F4 hybridoma clone is IgG2B/κ.

According to other preferred embodiments of the invention, the isotype of the antibody construct produced by the 6E3 hybridoma clone is IgG1/κ.

The terms “an antibody capable of binding FLNA” or “anti-FLNA antibody,” for example, refer to an antibody, or an antigen binding fragment thereof, that is capable of binding FLNA with sufficient affinity such that the antibody is useful as a diagnostic agent in targeting FLNA.

### G. Labeling

The invention provides a method for detecting FLNA in a biological sample comprising contacting a biological sample with an antibody, or antibody portion, of the invention and detecting either the antibody (or antibody portion) bound to FLNA or unbound antibody (or antibody portion), to thereby detect FLNA in the biological sample. The antibody is directly or indirectly labeled with a detectable substance to facilitate detection of the bound or unbound antibody.

Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

One skilled in the art will recognize that many strategies can be used for labeling target molecules to enable their detection or discrimination in a mixture of particles (e.g., labeled anti-FLNA antibodies as described herein). The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner, e.g. a FLNA antibody as described herein, that binds to FLNA, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule, e.g. a FLNA antibody as described herein, that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g. a FLNA antibody as described herein.

In some embodiments, the invention provides a label for the detection of FLNA, wherein the label comprises a binding partner for the marker and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an anti-FLNA antibody as described herein.

Alternative to labeling the antibody, FLNA can be assayed in biological fluids by a competition immunoassay utilizing FLNA standards labeled with a detectable substance and an unlabeled FLNA antibody. In this assay, the biological sample, the labeled FLNA standards and the FLNA antibody are combined and the amount of labeled standard bound to the unlabeled antibody is determined. The amount of FLNA in the biological sample is inversely proportional to the amount of labeled standard bound to the anti-FLNA antibody. Similarly, FLNA can also be assayed in biological fluids by a competition immunoassay utilizing FLNA standards labeled with a detectable substance and an unlabeled FLNA antibody.

### H. Detection of Expression Levels

Marker levels can be detected based on the absolute expression level or a normalized or relative expression level. Detection of absolute marker levels may be preferable when monitoring the treatment of a subject or in determining if there is a change in the abnormal prostate state status of a subject. For example, the expression level of one or more markers can be monitored in a subject being monitored following a negative biopsy, e.g., at regular intervals, such a monthly intervals. A modulation in the level of one or more markers can be monitored over time to observe trends in changes in marker levels. Expression levels of FLNA in the subject may be higher than the expression level of those markers in a normal sample, but may be lower than the prior expression level, thus indicating a benefit of a treatment regimen for the subject. Similarly, rates of change of marker levels can be important in a subject who is not subject to active treatment for prostate cancer or BPH (e.g., active monitoring following a negative biopsy). Changes, or not, in marker levels may be more relevant to treatment decisions for the subject than marker levels present in the population. Rapid changes in marker levels in a subject who otherwise appears to have a normal prostate may be indicative of an abnormal prostate state, even if the markers are within normal ranges for the population.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cancer sample, or between samples from different sources.

Alternatively, the expression level can be provided as a relative expression level as compared to an appropriate control, e.g., population control, adjacent normal tissue control, earlier time point control, etc. Preferably, the samples used in the baseline determination will be from cells from a subject that does not have an abnormal prostate state. The choice of the sample source is dependent on the use of the relative expression level. In addition, as more data is accumulated, the mean expression value can be revised, providing improved relative expression values based on accumulated data. Expression data from cancer cells provides a means for grading the severity of the cancer state.

As described in detail herein, the expression level of FLNA in a sample can be detected and/or quantified by using any one or more of the binding proteins described herein, wherein FLNA is detected and/or quantified under conditions such that the binding protein binds to FLNA in the sample.

In the methods of the invention, the level of FLNA can be detected using, for example, an enzyme-linked immunosorbent assay (ELISA), radioimmunoassay (RIA), antibody-labeled fluorescence imaging, tissue immunohistochemistry, or an immunoprecipitation-multiple reaction monitoring (IPMRM) assay, as described herein.

The present invention also provides methods for measuring the level of FLNA protein in a biological sample by detecting and/or quantifying the amount of one or more FLNA surrogate peptides in a protein digest prepared from the biological sample (e.g., protein digest prepared from FLNA protein isolated, purified or precipitated from the biological sample, e.g. by using a FLNA binding protein) using mass spectrometry; and calculating the level of FLNA protein in the sample. In one embodiment, the amount of FLNA is a relative amount or an absolute amount. In a particular embodiment, the protein digest comprises a protease digest, for example, a trypsin digest.

Quantifying the amount of one or more FLNA surrogate peptides may comprise comparing an amount of one or more FLNA surrogate peptides in one biological sample to the amount of the same FLNA surrogate peptides in a different and separate biological sample. Quantifying one or more FLNA surrogate peptides may comprise determining the amount of the each of the FLNA surrogate peptides in a biological sample by comparison to an added, corresponding internal standard peptide of known amount, where each of the FLNA surrogate peptides in the biological sample is compared to an internal standard peptide having the same amino acid sequence. The internal standard peptide may be an isotopically labeled peptide. The isotopically labeled internal standard peptide may contain one or more heavy stable isotopes selected from ¹⁸O, ¹⁷O, ³⁴S, ¹⁵N, ¹³N, ¹³C, ²H or combinations thereof.

In these embodiments, the mass spectrometry may comprise tandem mass spectrometry, ion trap mass spectrometry, triple quadrupole mass spectrometry, MALDI-TOF mass spectrometry, MALDI mass spectrometry, and/or time of flight mass spectrometry. The mode of mass spectrometry used may be, for example, Multiple Reaction Monitoring (MRM).

The present invention provides methods for detecting and/or quantifying the level of FLNA in a sample by detecting and/or quantifying one or more surrogate peptides comprising or consisting of the amino acid sequence of SEQ ID NO:35 (P2) and/or SEQ ID NO:36 (P4), in a protein digest prepared from the sample using a mass spectrometry technique, e.g., multiple reaction monitoring (MRM). In one embodiment, the protein digest is prepared from FLNA protein isolated, purified or precipitated from the biological sample, e.g. by using a FLNA binding protein such as a binding protein described herein. In one embodiment, the MRM is immunoprecipitation-multiple reaction monitoring (IPMRM) comprising a FLNA immunoprecipitation step, wherein the immunoprecipitation is carried out using one or more binding proteins as described herein.

In these embodiments, the one or more surrogate peptides detected by mass spectrometry, e.g., MRM, have amino acid sequences consisting of SEQ ID NO:35 and/or SEQ ID NO:36. In other embodiments, the surrogate peptide detected by mass spectrometry, e.g., MRM, has an amino acid sequence comprising SEQ ID NO:35. In a preferred embodiment, the surrogate peptide detected in the mass spectrometry assay, e.g., MRM, has an amino acid sequence consisting of SEQ ID NO:35.

In one embodiment, MRM comprises identifying the one or more surrogate peptides using one or more mass transitions m/z selected from the group consisting of: 441.7 (M+2H)²⁺→584.5 (y₅¹⁺) for P2; 535 (M+3H)³⁺→832.4 (y₈¹⁺) for P4, 445.5 (M+2H)²⁺→592.1 (y₅¹⁺) for P2 internal standard (P2_IS), and 538.4 (M+3H)³⁺→842.5 (y₈¹⁺) for P4 internal standard P4 IS. In one embodiment, MRM comprises detecting and/or quantifying a surrogate peptide for FLNA having the amino acid sequence consisting of SEQ ID NO:35 (P2) using the mass transition m/z 441.7 (M+2H)²⁺→584.5 (y₅¹⁺) for P2 and, optionally, further using the mass transition m/z 445.5 (M+2H)²⁺→592.1 (y₅¹⁺) for P2 internal standard (P2_IS). In one embodiment, MRM comprises detecting and/or quantifying a surrogate peptide for FLNA having the amino acid sequence consisting of SEQ ID NO:36 (P4) using the mass transition 535 (M+3H)³⁺→832.4 (y₈¹⁺) for P4 and, optionally, further using the mass transition 538.4 (M+3H)³⁺→842.5 (y₈¹⁺) for P4 internal standard P4_IS.

Methods for diagnosing an abnormal prostate state in a subject using an MRM assay, e.g., immunoprecipitation-multiple reaction monitoring (IPMRM), are also provided. In one embodiment, the level of FLNA in a biological sample from the subject is detected and compared with the level of FLNA in a normal control sample using MRM wherein one or more surrogate peptides comprising the amino acid sequence of SEQ ID NO:35 (P2) and/or SEQ ID NO:36 (P4) are detected, and wherein an altered level of FLNA in the biological sample relative to the normal control sample is indicative of an abnormal prostate state in the subject. An increased level of FLNA in the biological sample relative to the normal control sample is indicative of an abnormal prostate state, e.g., BPH or prostate cancer, in the subject, whereas no increase in the detected level of FLNA in the biological sample relative to the normal control sample is indicative of a normal prostate state in the subject.

In any of the foregoing embodiments, the immunoprecipitation step of the IPMRM can be carried out using any one or more of the binding proteins described herein. For example, the 2C12, 3F4, and/or the 6E3 antibodies may be used in the IPMRM methods of the invention. In one embodiment, the 2C12 and 3F4 antibodies are used in IPMRM. In one embodiment, the 2C12 antibody is used in IPMRM. In one embodiment, the 3F4 antibody is used in IPMRM. Also in any of the foregoing embodiments, the surrogate peptide detected in the assay can be P2 (SEQ ID NO: 35).

### I. Kits

The invention also provides compositions and kits for diagnosing, prognosing, or monitoring a disease or disorder, recurrence of a disorder, or survival of a subject being treated for a disorder (e.g., an abnormal prostate state, BPH, an oncologic disorder, e.g., prostate cancer). These kits include one or more of the following: a detectable antibody that specifically binds to a marker of the invention, reagents for obtaining and/or preparing subject tissue samples for staining, and instructions for use. In one embodiment, the antibody is any one or more of the binding proteins described herein, including the 2C12, 3F4, and/or 6E3 antibodies of the invention.

The invention also encompasses kits for detecting the presence of a marker protein or nucleic acid in a biological sample. Such kits can be used to determine if a subject is suffering from or is at increased risk of developing an abnormal prostate state, e.g., BPH, can be used for monitoring a subject suspected of having an abnormal prostate state, for differentiating between BPH and prostate cancer, and for avoiding unnecessary biopsy in a subject, using the biomarker panel of the invention, e.g., FLNA, age and prostate volume. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The instructions can provide instructions for detecting prostate volume and determining age of the subject, and analyzing the results to diagnose the subject.

The kits can also include reagents for detection of a control protein in the sample not related to the abnormal prostate state, e.g., actin for tissue samples, albumin in blood or blood derived samples for normalization of the amount of the marker present in the sample. The kit can also include the purified marker for detection for use as a control or for quantitation of the assay performed with the kit.

Kits include reagents for use in a method to diagnose prostate cancer in a subject (or to identify a subject predisposed to developing prostate cancer, etc.), the kit comprising a detection reagent, e.g. an antibody of the invention, wherein the detection reagent is specific for a prostate cancer-specific protein, e.g. FLNA. In one embodiment, the detection reagent is any one or more of the binding proteins described herein, including the 2C12, 3F4, and/or 6E3 antibodies of the invention. In one embodiment, the detection reagent comprises the 2C12 and/or 3F4 antibodies. In one embodiment, the detection reagent comprises the 2C12 antibody. In one embodiment, the detection reagent comprises the 3F4 antibody.

For antibody-based kits, the kit can comprise, for example: (1) a first antibody (e.g., attached to a solid support) which binds to a first marker protein; and, optionally, (2) a second, different antibody which binds to either the first marker protein or the first antibody and is conjugated to a detectable label. In certain embodiments, the kit includes (1) a second antibody (e.g., attached to a solid support) which binds to a second marker protein; and, optionally, (2) a second, different antibody which binds to either the second marker protein or the second antibody and is conjugated to a detectable label. The first and second marker proteins are different. In an embodiment, the first marker is FLNA. In another embodiment, either the first or the second marker is PSA. In other certain embodiments, neither the first marker nor the second marker is PSA. In certain embodiments, the kit comprises a third antibody which binds to a third marker protein which is different from the first and second marker proteins, and a second different antibody that binds to either the third marker protein or the antibody that binds the third marker protein wherein the third marker protein is different from the first and second marker proteins.

Reagents specific for detection of a marker of the invention, e.g., FLNA, allow for detection and quantitation of the marker in a complex mixture, e.g., serum, tissue sample. In certain embodiments, the reagents are species specific. In certain embodiments, the reagents are not species specific. In certain embodiments, the reagents are isoform specific. In certain embodiments, the reagents are not isoform specific. In certain embodiments, the reagents detect total FLNA.

In certain embodiments, the kit includes reagents for use in an immunoprecipitation assay for the detection of FLNA, wherein the immunoprecipitation assay is followed by a multiple reaction monitoring (MRM) assay. In one embodiment, labeled surrogate peptides for FLNA, e.g. P2 and/or P4, are included in the kit for use as internal standards.

In certain embodiments, the kits can also comprise any one of, but not limited to, a buffering agent(s), a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target markers. Each component of the kit can be enclosed within an individual container and all of the various containers can be within a single package, along with instructions for interpreting the results of the assays performed using the kit.

The kits of the invention may optionally comprise additional components useful for performing the methods of the invention.

### Example

### Materials and Methods

### Prostate Volume Calculation

Prostate volumes are calculated using the ellipse technique, L×W×H×0.52.

Measurements were taken as follows: width on axial view, length and height on sagittal view on a Philips iU22 with endfire C9-5 transducer (Philips, Bothell, WA).

### Serum Preparation

Serum separator tubes were kept ambient for 30 minutes following blood collection, stored at 4° C. during transport to the lab, and then promptly centrifuged at 1500×g for 15 minutes at 4° C. The serum was divided into four 500 μL aliquots in 2 mL cryovials and stored in vapor phase liquid nitrogen until use.

### Quantitation of FLNA Peptides by Immunoprecipitation and LC-MS/MS (MRM) Analysis

Antibody immobilization. Three mouse monoclonal antibodies, anti-FLNA 2C12, anti-FLNA 3F4, and anti-FLNA 6E3 (as described herein and in U.S. patent application Ser. No. 15/801,093, filed on Nov. 1, 2017, the contents of which are hereby incorporated herein by reference) were immobilized onto an agarose support using the ThermoFisher Scientific Pierce Direct IP Kit™ (ThermoFisher Scientific) according to the manufacturer's protocol with minor modifications. 200 μg of each of the three antibodies, were coupled individually to 200 μL of AminoLink Plus coupling resin and stored at 4° C. until needed.

Immunoprecipitation and preparation of calibration standards. Immunoprecipitation was performed using the Pierce Direct IP Kit™ (ThermoFisher Scientific) according to the manufacturer's protocol with minor modifications. Immunoprecipitation tubes were prepared by aliquoting 5 μL of each of the three antibody-coupled resins into the IP tube (Pierce Direct IP Kit™, ThermoFisher Scientific). The resin was washed twice with 200 μL of IP lysis/wash buffer. 100 μL of human serum sample or 100 μL of water (surrogate matrix) was added to each IP tube along with 500 μL of prepared lysis buffer solution (IP lysis/wash buffer with 1.2×Halt protease cocktail Inhibitor™; ThermoFisher Scientific) and 0.5M EDTA and incubated overnight at 4° C. with end-over-end mixing. The resin was washed five times with 200 μL of IP lysis/wash buffer and once with 100 μL of 1× conditioning buffer. The captured proteins were eluted with 50 μL of elution buffer with an incubation time of 15 minutes and neutralized with 5 μL of 1M Tris HCl, pH 9.0 (Teknova, Hollister, CA). The IP eluates from the surrogate matrix were used to prepare P2 (AGVAPLQVK) (SEQ ID NO: 35) and P4 (YNEQHVPGSPFTAR) (SEQ ID NO: 36) peptide calibration curves by spiking with a P2/P4 synthetic peptide (Genscript, Piscataway, NJ) stock solution (0.2/0.36 μg/mL) followed by serial dilution. P2 and P4 calibration standards ranged from 125 pg/mL to 2000 pg/mL, and 1125 pg/mL to 36000 pg/mL, respectively. All samples were then subjected to trypsin digestion as described below.

Digestion of IP-extracted samples using trypsin. Trypsin digestion was performed using the Flash Digest Kit™ (Perfinity Biosciences, West Lafayette, IN) following the manufacturer's protocol with minor modifications. Flash digest tubes were equilibrated to room temperature and then centrifuged for 1 min at 1500×g and 5° C. 50 μL of each sample, 25 μL of digestion buffer (Perfinity Biosciences), and 5 μL of working internal standard (ThermoFisher Scientific) solution (P2/P4 10/30 ng/ml) were added to the Flash™ digest tubes. After vortexing, samples were digested at 70° C. for 20 minutes in the Eppendorf, ThermoMixer C™ (Eppendorf). The Flash™ digest tubes were then centrifuged for 5 minutes at 1500×g and 5° C. A 60 μL aliquot of the supernatant was transferred to an LC-MS vial.

### LC-MS/MS (MRM) Analysis

MRM analyses were performed on a 6500 QTRAP™ mass spectrometer (Sciex) equipped with an electrospray source, a 1290 Infinity UPLC™ system (Agilent Technologies, Santa Clara, CA), and a XBridge® Peptide BEH300 C18™ (3.5 μm, 2.1 mm×150 mm) column (Waters, Milford, MA). Liquid chromatography was carried out at a flow rate of 400 μL/min, and the sample injection volume was 30 μL. The column was maintained at a temperature of 60° C. Mobile phase A consisted of 0.1% formic acid (Sigma Aldrich) in water (ThermoFisher Scientific) and mobile phase B consisted of 0.1% formic acid in acetonitrile (ThermoFisher Scientific). The gradient with respect to % B was as follows: 0-1.5 min, 5%; 1.5-2 min, 5-15%; 2-5 min, 15%; 5-7.1 min, 15-20%; 7.1-8.1 min, 20-80%; 8.1-9.0 min, 80%; and 9.0-9.1 min, 80-5%, 9.1-16 min, 5%. The instrument parameters for 6500 QTRAP™ mass spectrometer were as follows: Ion spray voltage of 5500 V, curtain gas of 20 psi, collision gas set to “medium”, interface heater temperature of 400° C., nebulizer gas (GS1) of 80 psi and ion source gas (GS2) of 80 psi, and unit resolution for both Q1 and Q3 quadrupoles.

### IPMRM Data Analysis and Quantitation

Data analysis was performed using the Analyst® software (version 1.6.2, AB Sciex, Framingham, MA) and peak integrations were reviewed manually. The calibration curve for FLNA P2 and P4 peptides was constructed by plotting the peak area ratios (analyte/internal standard) versus concentration of the standard with 1/x2 linear least square regression. The regression equations from P2 and P4 calibration standards were used to back-calculate the measured P2 and P4 concentrations for each QC and unknown sample.

### Statistical Analysis

Regression models were built and compared for their ability to classify patients with prostate cancer with low Gleason score (≤7), high Gleason score (≥8), and with an absence of cancer on biopsy. The resulting biomarker panel predictive algorithms were based on the regression models and probability threshold values selected to achieve a certain level of test sensitivity or specificity. All analyses were performed in R 3.2.2 with a significance level of 0.05, unless otherwise stated.

### Results

In order to identify and assess the clinical utility of serum biomarkers for PCa, biobank samples collected by CPDR/Walter Reed, University of Toronto/UHN, Duke University/Veterans affairs (VA), and Cleveland Clinic were retrospectively analyzed. Patient demographics of the samples used are shown in Table 2, below. There are no differences between patients with BPH and patients with PCa in age, PSA, number of biopsies etc. (Table 2). In addition, the distribution of race and sampling of patients from each clinical site used in this study are similar between patient groups with BPH and PCa (Table 2).

TABLE 2
Patient Demographics

| | BPH | PCa |
|---|---|---|
| | Mean ± SD | Mean ± SD |
| Age | 64.6 ± 8.5 | 61.9 ± 8 |
| PSA | 6.7 ± 4.7 | 6.5 ± 5.9 |

| | n | (% Total) | n | (% Total) |
|---|---|---|---|---|
| Race | | | | |
| Caucasian-American | 191 | (64%) | 281 | (59%) |
| African-American | 48 | (16%) | 139 | (29%) |
| Other | 61 | (20%) | 57 | (12%) |
| Clinical Site | | | | |
| CPDR/Walter Reed | 119 | (40%) | 239 | (50%) |
| Veteran Affairs | 58 | (19%) | 34 | (7%) |
| UHN | 118 | (39%) | 78 | (16%) |
| CCF | 5 | (2%) | 126 | (26%) |
| Total | 300 | | 477 | |

Age (years) and PSA levels (ng/mL) (Mean ± standard deviation, SD). Distribution of patients by race and sampling distribution of patients from various clinical sites.

Age, prostate volume, FLNA levels, and PSA levels were first compared between patients who have had one or more biopsy and were diagnosed with BPH or PCa (FIG. 1A). There was no significant difference between groups for each factor alone.

Regression modeling analysis was then used to identify the best set of predictive factors. Irrespective of number of biopsies, the combination of the factors age, prostate volume, and FLNA level was found to have better predicative performance than PSA in discriminating BPH from PCa (AUC 0.75 vs. 0.52 for PSA; FIG. 1B, Table 3).

As sensitivity is critical in identifying the true positive rate of a diagnostic test, the aim was to utilize a biomarker panel with diagnostic sensitivity ≥0.9. This resulted in a cutoff for the model at 0.498, and yielded a specificity of 0.43 with positive and negative predictive values of 0.72 and 0.73, respectively (Table 3). The diagnostic odds ratio (OR) for the biomarker panel in predicting PCa in all patients who have had biopsy is 7.0 (95% CI 4.85, 10.2, Table 3). Moreover, the performance of the biomarker panel indicates that in the current study cohort, 170 (57%) patients without PCa would be recommended for biopsy, which is a 43% reduction in unnecessary biopsies compared to 300 patients with PSA. This model predicts that only 47 (10%) of patients with PCa would not be recommended to have a biopsy. To potentially reduce the likelihood for multiple biopsies, a diagnostic “adjuvant” test should have good performance in discriminating BPH from PCa in patients who have had multiple biopsies (two or more biopsies). For each of the factors in the biomarker panel alone, no statistically significant differences are observed between patients with BPH and PCa in patients who have had more than one biopsy (FIG. 2A). However, when the factors are combined (i.e., age, prostate volume, and FLNA levels) this yielded a diagnostic performance that is improved over that of PSA in discriminating patients with BPH from PCa in patients who have had multiple biopsies (AUC 0.87 vs. 0.52 for PSA; FIG. 2B; Table 3).

In addition, diagnostic sensitivity set at 0.9 yields a specificity of 0.67 with positive and negative predictive values of 0.93 and 0.58, respectively (Table 3). The diagnostic OR of the biomarker panel in patients who had multiple biopsies is 18.9 (95% CI 11.2, 31.9; Table 3). The performance of the biomarker panel in patients who have had multiple biopsies indicates that 31 (33%) patients without PCa in this study cohort would be recommended to get a biopsy. Thus, compared to PSA, the biomarker panel would reduce the number of biopsy recommendations by 67% in patients without PCa. This would result in only 47 (10%) patients with PCa that would not receive a recommendation for biopsy.

TABLE 3
Clinical Utility of Biomarker Panel compared to PSA
in Patients who have had Biopsy or Multiple Biopsies.

| Model | AUC | Sensitivity | Specificity | PPV | NPV | OR | (95% CI) |
|---|---|---|---|---|---|---|---|
| 1 or | 0.75 | 0.9 | 0.43 | 0.72 | 0.73 | 7.0 | (4.8, |
| More | | | | | | | 10.2)* |
| Biopsy | | | | | | | |
| Multiple | 0.87 | 0.9 | 0.67 | 0.93 | 0.58 | 18.9 | (11.2, |
| Biopsy | | | | | | | 31.9)* |

Diagnostic performance (AUC), sensitivity, specificity, positive predictive value (PPV) and negative predictive value (NPV), odds ratio (OR), and 95% confidence interval (CI) of the OR for each model.
*p < 0.05 in discrimination accuracy.

These findings indicate that the use of the biomarker panel prior to biopsy improves the selection of men for biopsy, in addition to reducing the need for biopsy and unnecessary harm from intervention in patients with BPH.

The biomarker panel's ability to discriminate BPH from PCa in patients with an intermediate Gleason score (5-7), and those with a high score (≥8) was also assessed. Results indicate a diagnostic performance that is improved over that of PSA in discriminating patients with BPH from PCa in patients with an intermediate Gleason score (5-7) (AUC 0.76 vs. 0.56 for PSA; FIG. 3A, Table 4). In addition, diagnostic sensitivity set at 0.9 yields a specificity of 0.44 with positive and negative predictive values of 0.72 and 0.66, respectively (Table 4). The diagnostic OR of the biomarker panel in patients who had multiple biopsies is 7.2 (95% CI 4.9, 10.6; Table 4).

Furthermore, the biomarker panel's performance in discriminating BPH from patients with more aggressive PCa, Gleason (8-10), was also found to be better than PSA (AUC 0.74 vs. 0.47 for PSA; FIG. 3B, Table 4). Moreover, diagnostic sensitivity set at 0.9 yields a specificity of 0.45 and positive and negative predictive values of 0.18 and 0.97, respectively (Table 4). The diagnostic OR of the biomarker panel is 7.5 (95% CI 2.6, 21.5) in discriminating patients with BPH from those with aggressive PCa (Table 4).

TABLE 4
Clinical Utility of Biomarker Panel to discriminate BPH
from PCa in patients with an intermediate Gleason score (5-7),
and those with a high score (≥ 8).

| Model | AUC | Sensitivity | Specificity | PPV | NPV | OR | (95% CI) |
|---|---|---|---|---|---|---|---|
| BPH vs | 0.76 | 0.9 | 0.44 | 0.72 | 0.66 | 7.2 | (4.9, 10.6) |
| PCa | | | | | | | |
| Gleason | | | | | | | |
| <7 | | | | | | | |
| BPH vs | 0.74 | 0.9 | 0.45 | 0.18 | 0.97 | 7.5 | (2.6, 21.5) |
| PCa | | | | | | | |
| Gleason | | | | | | | |
| 8-10 | | | | | | | |

Diagnostic performance (AUC), sensitivity, specificity, positive predictive value (PPV) and negative predictive value (NPV), odds ratio (OR), and 95% confidence interval (CI) of the OR for each model.
*p < 0.05 in discrimination accuracy.

The biomarker panel's ability to discriminate BPH from PCa in patients with a Gleason score of 5-6, and those with a Gleason score of 7-10 was also assessed.

Results indicate a diagnostic performance that is improved over that of PSA in discriminating patients with BPH from PCa in patients with a Gleason score of 5-6 (AUC 0.77 vs. 0.61 for PSA; FIG. 4A).

Furthermore, the biomarker panel's performance in discriminating BPH from patients with more aggressive PCa, Gleason 7-10, was also found to be better than PSA (AUC 0.8 vs. 0.52 for PSA; FIG. 4B).

### DISCUSSION

This Example describes the analysis of the combinatorial power of a biomarker panel comprising filamin-A (FLNA), age, and prostate volume to predict clinical segregation of BPH versus PCa in 777 patients. Retrospective analysis of biobank samples from patients with LUT/BPH and patients with PCa was conducted as described above, with results indicating a diagnostic performance that is improved over that of PSA in discriminating patients with BPH from patients with PCa, irrespective of the number of biopsies, and in discriminating patients with BPH from PCa in patients with an intermediate Gleason score (>5) or an aggressive Gleason score (>7).

There were no differences in age, PSA levels, number of biopsies etc. in both sets of patients (BPH vs. PCa), and the distribution of race and sampling of patients from each clinical site used in the study are similar between patient groups. Thus, the biomarker panel described herein, e.g., FLNA levels, age and prostate volume, can be used to discriminate between BPH and prostate cancer, diagnose BPH, and avoid unnecessary, potentially harmful and costly biopsies.

| Sequence Identifier | Protein or Nucleic Acid |
|---|---|
| SEQ ID NO: 1 | 2C12 variable heavy (VH) domain |
| SEQ ID NO: 2 | 2C12 variable light (VL) domain |
| SEQ ID NO: 3 | 3F4 variable heavy (VH) domain |
| SEQ ID NO: 4 | 3F4 variable light (VL) domain |
| SEQ ID NO: 5 | 6E3 variable heavy (VH) domain |
| SEQ ID NO: 6 | 6E3 variable light (VL) domain |
| SEQ ID NO: 7 | 2C12 VH CDR1 |
| SEQ ID NO: 8 | 2C12 VH CDR2 |
| SEQ ID NO: 9 | 2C12 VH CDR3 |
| SEQ ID NO: 10 | 2C12 VL CDR1 |
| SEQ ID NO: 11 | 2C12 VL CDR2 |
| SEQ ID NO: 12 | 2C12 VL CDR3 |
| SEQ ID NO: 13 | 3F4 VH CDR1 |
| SEQ ID NO: 14 | 3F4 VH CDR2 |
| SEQ ID NO: 15 | 3F4 VH CDR3 |
| SEQ ID NO: 16 | 3F4 VL CDR1 |
| SEQ ID NO: 17 | 3F4 VL CDR2 |
| SEQ ID NO: 18 | 3F4 VL CDR3 |
| SEQ ID NO: 19 | 6E3 VH CDR1 |
| SEQ ID NO: 20 | 6E3 VH CDR2 |
| SEQ ID NO: 21 | 6E3 VH CDR3 |
| SEQ ID NO: 22 | 6E3 VL CDR1 |
| SEQ ID NO: 23 | 6E3 VL CDR2 |
| SEQ ID NO: 24 | 6E3 VL CDR3 |
| SEQ ID NO: 25 | 2C12 hybridoma clone heavy chain consensus |
| SEQ ID NO: 26 | 2C12 hybridoma clone light chain consensus |
| SEQ ID NO: 27 | 3F4 hybridoma clone heavy chain consensus |
| SEQ ID NO: 28 | 3F4 hybridoma clone light chain consensus |
| SEQ ID NO: 29 | 6E3 hybridoma clone heavy chain consensus |
| SEQ ID NO: 30 | 6E3 hybridoma clone light chain consensus |
| SEQ ID NO: 31 | Filamin A, isoform 1, nucleotide |
| SEQ ID NO: 32 | Filamin A, isoform 1, protein |
| SEQ ID NO: 33 | Filamin A, isoform 2, nucleotide |
| SEQ ID NO: 34 | Filamin A, isoform 2, protein |
| SEQ ID NO: 35 | peptide |
| SEQ ID NO: 36 | peptide |

1.-96. (canceled)

97. A method for treating benign prostatic hyperplasia (BPH), comprising:
(a) detecting the protein level of Filamin A (FLNA) in a biological sample from a subject;
(b) obtaining measurement of prostate volume of the subject;
(c) producing a score on the basis of the detected level of FLNA in the sample in combination with the age and prostate volume of the subject, wherein the score is not produced on the basis of the level of keratin 19 (KRT19);
(d) identifying the subject to have BPH when the score is below a threshold score; and
(e) administering to the subject a therapeutic treatment for BPH, wherein the therapeutic treatment comprises a selective al-blocker, a 5α-reductase inhibitor, an antimuscarinic, a phosphodiesterase-5 inhibitor, a surgery, a prostatic stent, a high intensity focused ultrasound, an interstitial laser coagulation, a transurethral electroevaporation of the prostate, a transurethral microwave thermotherapy, a transurethral needle ablation, a photoselective vaporization, or a combination thereof.

98. The method of claim 97, wherein if the score is above the threshold score, the subject is diagnosed with prostate cancer.

99. The method of claim 97, wherein the protein level of FLNA is detected using an assay selected from an immunoassay, an enzyme-linked immunosorbent assay (ELISA), immunoprecipitation multiple reaction monitoring (IP-MRM), or a mass spectrometry assay.

100. The method of claim 97, wherein the biological sample is selected from the group consisting of blood, serum, plasma, and urine.

101. The method of claim 97, wherein the age of the subject is 50 years or older, the subject is experiencing lower urinary tract symptoms (LUTS), the subject has an enlarged prostate gland as determined by digital rectal examination (DRE), and/or the subject has an elevated prostate specific antigen (PSA) level.

102. The method of claim 97, wherein the subject does not have an enlarged prostate gland as determined by digital rectal examination (DRE).

103. The method of claim 97, wherein the subject has had one or more prostate biopsies.

104. The method of claim 97, wherein the BPH is differentiated from prostate cancer in a subject having an intermediate Gleason score of from 5 to 7, or a high Gleason score of greater than 8.
